(19) United States
(12) Patent Application Publication
(10) Pub. No.: US 2016/0256388 A1
(43) Pub. Date: Sep. 8, 2016

(54) COMPOSITIONS AND METHODS FOR TREATMENT OF RESPIRATORY TRACT INFECTIONS

(71) Applicants: Uri Galili, Chicago, IL (US); Haruko Ogawa, Obihiro (JP)

(72) Inventors: Uri Galili, Chicago, IL (US); Haruko Ogawa, Obihiro (JP)

(21) Appl. No.: 14/998,906

(22) Filed: Mar. 2, 2016

Related U.S. Application Data
(60) Provisional application No. 62/177,115, filed on Mar. 5, 2015.
(60) Provisional application No. 62/230,321, filed on Jun. 2, 2015.

(51) Int. Cl. A61K 9/127; A61K 38/17; A61K 31/7004; A61K 9/00

(52) U.S. Cl. CPC A61K 9/1271; A61K 9/007; A61K 38/177; A61K 31/7004

(57) ABSTRACT

This invention teaches a novel treatment of patients infected with influenza virus in early stages of the disease, with liposomes called α-gal/SA liposomes, in order to decrease the infection period and decrease further complications by this disease. The treatment is based on inhalation of biodegradable liposomes that present two types of carbohydrate epitopes: α-Gal epitopes with the structure Galα1-3Galβ1-4(3)GlcNAc-R) and sialic acid (SA) epitopes. The treatment is based on the ability of influenza virus to bind to SA epitopes and on the binding of the natural anti-Gal antibody (the most abundant natural antibody in humans) to α-gal epitopes. Following inhalation of aerosolized α-gal/SA liposomes they land in the mucus lining the respiratory tract. The α-gal/SA liposomes bind influenza virus via SA epitopes interaction with hemagglutinin of the virus, thus they slow or prevent the progress of the influenza virus infection process. Binding of the natural anti-Gal antibody to α-gal epitopes on α-gal/SA liposomes causes complement mediated chemotactic recruitment of macrophages and dendritic cells which internalize via Fc/Fc receptor interaction the α-gal/SA liposomes and the influenza virus bound to them and destroy this virus. The recruited macrophages and dendritic cells further process the immunogenic peptides of the internalized virus, transported them to the regional lymph nodes and present these peptides for eliciting an effective protective immune response that ends the influenza virus infection in a period shorter than in untreated patients and prevents further complications in the respiratory system and in other parts of the body.

### CROSS REFERENCE TO RELATED APPLICATIONS

This application claims the benefit of U.S. provisional patent application 62/177,115 entitled “COMPOSITIONS AND METHODS FOR TREATMENT OF PATIENTS WITH RESPIRATORY TRACT INFECTIONS” and filed by Uri Galili on Mar. 5, 2015 and of U.S. provisional patent application 62/230,321 entitled “COMPOSITIONS AND METHODS FOR TREATMENT OF BIRDS WITH RESPIRATORY TRACT INFECTIONS” and filed by Uri Galili on Jun. 2, 2015, the contents of which are incorporated in this application.

### FIELD OF THE INVENTION

The present invention relates to the field of treatment of respiratory tract infections in general and influenza virus infections in particular. In one embodiment, the present invention provides compositions and methods for treatment of influenza (commonly known as “flu”) patients by inhalation of liposomes that present both α-gal epitopes (Galα1-3Galβ1-4(3)GlcNAc-R) and sialic acid (SA) epitopes (referred to as α-gal/SA liposomes). When administered as aerosol by inhalation into patients or into birds at the early stages of influenza virus infection (i.e., when the influenza patient becomes symptomatic), the influenza virus binds to the SA epitopes on the α-gal/SA liposomes and thus is prevented from infecting the epithelium lining the respiratory tract. The α-gal/SA liposomes also induce rapid recruitment and migration of macrophages and dendritic cells toward the inhaled α-gal/SA liposomes trapped in the mucus layer lining the respiratory tract. The α-gal/SA liposomes further induce effective uptake and destruction of the influenza virus bound to the α-gal/SA liposomes by macrophages and dendritic cells and thus decrease the virus burden in the respiratory tract. The macrophages and dendritic cells also function as antigen presenting cells (APC) that transport the influenza virus antigens to the regional lymph nodes and rapidly induce humoral and cellular immune responses that effectively protect the treated patient or treated bird against the infecting influenza virus. In another embodiment, the present invention provides for a method of treatment by liposomes presenting α-gal epitopes and “docking” receptors of other respiratory pathogens.

### BACKGROUND OF THE INVENTION

Influenza (flu) is a contagious respiratory disease caused by influenza virus infection. Annual influenza outbreaks in the United States affect 5-20% of the population (CDC Fact Sheet, 2006). Influenza spreads around the world in a yearly outbreak, resulting in about three to five million cases of severe illness and about 250,000 to 500,000 deaths (“Influenza (Seasonal) Fact sheet No 211”. who. int. March 2014). Influenza complications such as bacterial pneumonia, ear and/or sinus infections, dehydration and worsening of chronic medical conditions can result in severe illness and even death. Yearly influenza vaccinations are recommended for preventing the influenza disease, particularly for high-risk individuals (e.g., children, elderly, etc.) and their caretakers (e.g., health care workers).

Currently used inactivated influenza (flu) virus vaccines are the product of the 2+6 re-assortment containing hemagglutinin (HA) and neuraminidase (NA) genes from the vaccine target strain and the remaining genes from A/Puerto Rico/8/34-H1N1 (PR8) influenza virus strain, respectively. These vaccines display suboptimal efficacy as determined by the finding that approximately 25%-50% of immunized individuals (in particular elderly populations) contract the disease during the influenza season (Webster, Vaccine, 18: 1686, 2000). The virus is spread from an infected patient to healthy individuals by microdroplets (aerosol) carrying the virus and is distributed as a result of sneezing coughing or talking. The virus penetrating the upper and lower airways binds to sialic acid (SA) epitopes functioning as receptors on ciliated respiratory epithelium cells via the hemagglutinin (HA) protein on the virus, in mammals (Unverzagt et al. Carbohydr Res. 251: 285, 1994) and birds (Thompson et al. J Virol 80: 8060, 2006). The influenza virus bound to SA epitopes further penetrates into the cells by an endosome and releases its RNA-8 genetic pieces. After multiplication within infected cells, the core structure is covered by the cell membrane containing HA and neuraminidase (NA). The full virus detaches from the cell following the activity of viral NA that releases the virus from the contact with cell surface SA epitopes.

From the time of infection by influenza virus there is a “race” between the virus produced in increasing numbers in cells of the respiratory tract epithelium and the immune system that is activated to generate protective humoral and cellular immune responses against the infecting virus. Slowing the infection (i e inhibition of virus growth) is critical at the early stages of the infection in order to enable the immune system to mount a timely combination of humoral and cellular protective immune responses that prevent further increase in the virus burden. The humoral immune response is comprised primarily of production of anti-influenza virus IgA antibodies and to a lesser extent IgG antibodies that neutralize the virus and prevent further infection of healthy cells. The cellular immune response is comprised primarily of influenza virus specific T cells that kill virus infected cells, thereby contributing to prevention of further virus infection of healthy cells.

If the protective immune response is not induced fast enough, the virus burden will reach a size that is detrimental to the health of the infected individual because of extensive destruction of the respiratory epithelium and the facilitation of bacterial infections of the lungs, leading to possible lethal bacterial pneumonia. This scenario may be observed in children and in elderly individuals who succumb to the disease. It is assumed that by slowing infectivity of influenza virus in the respiratory epithelium, the infected patient may have more time to mount an effective anti-viral immune response and thus to overcome the infection and avoid detrimental effects of influenza. In attempt to slow virus growth at the early stages of influenza virus infection the FDA approved the use of 3 types of neuraminidase inhibitors: 1. Oseltamivir (Tamiinfluenza®) taken orally, 2. Zanamivir (Relenza®) taken by inhalation, and 3. Peramivir (Rapivab®) administered intravenously. By inhibiting the viral neuraminidase activity, these drugs aim to inhibit the release of newly formed influenza virions from the surface of infected cells. The efficacy of these neuraminidase inhibitor drugs in inducing an effective slowing of the influenza virus infection is still controversial since some clinical studies reported no beneficial effects whereas others reported some clinical effects.

The present invention teaches a novel method for slowing and possibly preventing further infection by influenza virus in early stages of influenza virus infection by inhaling α-gal/sialic acid liposomes (α-gal/SA liposomes). These liposomes bind the influenza virus on the surface of the respiratory epithelium and target it for destruction by recruited macrophages. Macrophages as well as dendritic cells are recruited as a result of anti-Gal antibody interaction with its ligand the α-gal epitope on α-gal glycolipids of the α-gal/SA liposomes (Galili et al. J Immunol 178: 4676, 2007; Wigglesworth et al. J Immunol 186: 4422, 2011). Anti-Gal is the most abundant natural antibody in humans constituting ˜1% of immunoglobulins (Galili et al. J Exp Med 160: 1519, 1984). The macrophages and dendritic cells that are recruited, internalize the infecting influenza virus bound to the α-gal/SA liposomes, destroy the virus and transport the viral antigens to the regional lymph nodes for effective stimulation of the immune system to mount protective humoral and cellular immune responses against the virus. Ultimately, this treatment may attenuate the severity of influenza virus infection and decrease morbidity and mortality from the disease because of the rapid and effective generation of a protective immune response against the influenza virus. For this purpose the invention exploits the need of influenza virus to bind to sialic acid epitopes (SA epitopes) on cell membranes in order to infect the cells. Following inhalation of α-gal/SA liposomes, these liposomes land in the mucus and surfactant lining the respiratory epithelium and bind the influenza virus via SA epitopes on the α-gal/SA liposomes. The invention further exploits the natural anti-Gal antibody, which is the most abundant antibody in all humans (Galili, Immunology 140: 1, 2013). Anti-Gal binds to α-gal epitopes on the α-gal/SA liposomes, induces local complement activation, followed by recruitment of macrophages and dendritic cells. The recruited macrophages and dendritic cells internalized these liposomes and the influenza virus bound to them as a result of interaction between the Fe portion of anti-Gal IgG antibody bound to the α-gal/SA liposomes and Fcγ receptors (FcγR) on these cells and interaction between the Fc portion of anti-Gal IgA antibody bound to the α-gal/SA liposomes and Fcα receptors (FcαR) on these cells. Binding of C3b deposited on α-gal/SA liposomes to C3b receptors on macrophages and dendritic cells further contribute to the internalization of liposomes and influenza virus bound to them by these cells. These macrophages and dendritic cells further function as antigen presenting cells (APC) transporting, processing and presenting the influenza virus immunogenic peptides to the immune system cells in the regional lymph nodes, thereby eliciting an effective and protective anti-influenza virus immune response that stops the progress of the infection.

### SUMMARY OF THE INVENTION

The present invention relates to the field of treatment of microbial infections in general and influenza virus infection in particular. In one embodiment this invention teaches how to treat patients infected with influenza virus in early stages of the disease in order to shorten the infection time, decrease morbidity and mortality and elicit a rapid protective immune response in the patient against the infecting influenza virus. In another embodiment this invention teaches how to treat birds such as, but not limited to chicken and ducks infected with influenza virus in early stages of the disease in order to shorten the infection time, decrease morbidity and mortality and elicit a rapid protective immune response in the treated bird against the infecting influenza virus. In one embodiment, the present invention provides compositions and methods for preparation of biodegradable liposomes that present multiple carbohydrate epitopes of two types: 1. α-Gal epitopes with the structure Galα1-3Galβ1-4(3)GlcNAc-R) where R is a carbohydrate chain or any linker linked to lipids, glycolipids, glycoproteins, proteoglycans or any polymer. 2. Sialic acid epitopes (called SA epitopes) in which sialic acid (SA) is linked to carbohydrate chains or any linker linked to lipids, glycolipids, glycoproteins, proteoglycans, or any polymer. The liposomes presenting multiple α-gal epitopes and SA epitopes are referred to as α-gal/SA liposomes. In one embodiment the present invention teaches how to treat patients and/or birds infected with influenza virus in early stages of the disease by inhalation of aerosolized α-gal/SA liposomes.

The present invention is based on two physiologic phenomena: 1. Influenza virus binds to SA epitopes on the cell membrane of the respiratory tract epithelium in order to infect these cells and proliferate in them, thus causing the influenza disease. 2. The natural anti-Gal antibody which is the most abundant natural antibody in all humans constituting ˜1% of immunoglobulin in IgG, IgA and IgM classes binds specifically α-gal epitopes. These two phenomena are part of the proposed method for treating patients infected with influenza virus. In one non-limiting example of α-gal/SA liposomes preparation, glycolipids carrying α-gal epitopes (called here α-gal glycolipids), glycolipids carrying SA epitopes (called here SA glycolipids) and phospholipids are dissolved and mixed in an organic solvent as known to those skilled in the art. Non-limiting examples of representative α-gal glycolipids, SA glycolipids and phospholipids are illustrated in FIG. 1. These mixed materials are dried together by methods known to those skilled in the art, then sonicated in saline or in other physiologic buffers to form liposomes that carry both α-gal epitopes and SA epitopes (i.e., α-gal/SA liposomes) as illustrated in FIG. 3. These α-gal/SA liposomes are used for treatment of patients infected by influenza virus. The α-gal/SA liposomes in the form of an aerosol are administered by inhalation to the airways of patients infected by influenza virus. The inhaled α-gal/SA liposomes are trapped in the mucus film lining the respiratory epithelium and in the surfactant film in the alveoli. These α-gal/SA liposomes slow or prevent the progress of the influenza virus infection process. Although knowledge of the mechanism(s) involved is not required in order to make and use the present invention, it is contemplated that the protective effects of the α-gal/SA liposomes against infecting influenza virus are mediated by the following sequential steps, which are also illustrated in FIG. 2: 1. Influenza virus within the mucus and surfactant lining the respiratory tract binds to the multiple SA epitopes on the α-gal/SA liposomes trapped within these mucus and surfactant and thus is prevented from further infection of cells of the airways. 2. The natural anti-Gal antibody binds to the multiple α-gal epitopes on the α-gal/SA liposomes and activates the complement system. This complement activation comprises production of chemotactic complement cleavage peptides such as C5a, C4a and/or C3a. 3. Monocyte, macrophage and dendritic cells are recruited by the complement cleavage chemotactic peptides toward the inhaled α-gal/SA liposomes. 4. The α-gal/SA liposomes with the bound influenza virus are internalized by the macrophages and dendritic cells as a result of interaction between the anti-Gal antibody immunocomplexed to α-gal/SA liposomes and Fc receptors (FcR) on the macrophages and dendritic cells; influenza virus is killed within these macrophages and dendritic cells which further function as antigen presenting cells (APC) that process the immunogenic influenza virus antigens and transport them to the draining lymph nodes. 5. The macrophages and dendritic cells present the processed influenza virus antigenic peptides to the T cells within the regional lymph nodes and thus elicit protective humoral and cellular immune responses. Step #5 is not illustrated in FIG. 2. The anti-Gal mediated effective uptake of influenza virus bound to α-gal/SA liposomes into macrophages decreases the virus burden in the respiratory tract and thus decreases the damage to the respiratory tract epithelium and slows the expansion of the virus in infected patient. The rapid and effective processing and transport of the influenza virus immunogenic peptides to the regional lymph nodes further result in the mounting of a relatively fast and effective immune response that stops the infection and enables the patient's immune system to overcome the influenza virus infection.

Since birds also produce the natural anti-Gal antibody (McKenzie et al. Transplantation 67:864, 1997; Cotter et al. Poult Sci. 84:220, 2005; Cotter and Van Eerden Poult Sci. 85:435, 2006; Minozzi et al. BMC Genet. 9:5, 2008) and since influenza virus binds to SA epitopes on bird respiratory epithelium (Thompson et al. J Virol supra, 2006), it is contemplated that inhalation of α-gal/SA liposomes by birds infected with influenza virus will have therapeutic anti-influenza virus effects as those described in FIG. 2 in human patients infected with influenza virus and treated with α-gal/SA liposomes.

In another embodiment the invention describes the possible treatment of other respiratory microbial infections by the use of liposomes presenting multiple α-gal epitopes and which also present carbohydrate receptors and other receptors for specific pathogens. This treatment will affect the pathogen by processes similar to those described above for treatment of influenza virus infection with the difference that the pathogen binds to the liposomes via interaction with its corresponding receptor presented on the liposomes. The internalization of the pathogen bound to the α-gal liposomes by macrophages and dendritic cells will be mediated by anti-Gal bound to α-gal epitopes on the liposomes by a process similar to that of the targeting of influenza virus for internalization by macrophages and dendritic cells via Fc/Fc receptors interaction, as described in step #4 above and in FIG. 2.

### DESCRIPTION OF THE FIGURES

FIG. 1 illustrates non-limiting exemplary carbohydrate epitopes linked to glycoproteins or glycolipids which are discussed in the present invention. A. Sialic acid (SA) epitopes (SA epitope) linked to a N-linked carbohydrate chain on a glycoprotein. The SA epitopes have the structure SAα2-6(3)Galβ1-4GlcNAc-R. Glycoproteins with SA may carry one or more SA epitopes. The N-linked carbohydrate chain is found in glycosylation sites comprised by amino acid sequences of asparagine (N) followed by any amino acid (-X) followed by serine or threonine (-S/T), i.e., N—X—S/T. The terminal SA is linked to the penultimate galactose (Gal) via α2-6 or α2-3 linkage and may display other linkages, as well with various penultimate units. B. Sialic acid (SA) epitope linked to a glycolipids. The SA epitope has the structure SAα2-6(3)Galβ1-4GlcNAc-R. The terminal SA is linked to the penultimate galactose (Gal) via a 2-6 or a 2-3 linkage and may display other linkages to galactose (Gal) or to N-acetylgalactosamine (GalNAc) or to other penultimate units. Glycolipids with SA may carry one or more SA epitopes at the non-reducing ends and are also referred to as SA-glycolipids or gangliosides. C. α-Gal epitopes linked to an N-linked carbohydrate chain on a glycoprotein. The α-gal epitopes has the structure Galα1-3Galβ1-4GlcNAc-R. The terminal galactose (Gal) of the α-gal epitope is linked to the penultimate galactose (Gal) via α1-3 linkage and may display other linkages, as well as other penultimate units. Glycoproteins carry one or more α-gal epitopes on each carbohydrate chain. D. α-Gal epitope linked to a glycolipid. The α-gal epitope has the structure Galα1-3Galβ1-4GlcNAc-R. The terminal galactose (Gal) is linked to the penultimate galactose (Gal) via α1-3 linkage and may display other linkages, as well as other penultimate units. Glycolipids may carry one or more α-gal epitopes at the non-reducing ends of their carbohydrate chain and are also referred to as α-gal glycolipids. α-Gal epitopes bind the natural anti-Gal antibody which is abundant in humans.

FIG. 2 illustrates some of the sequential processes (steps) occurring after the inhaled α-gal/SA liposomes land in the mucus and surfactant lining the respiratory tract: 1. Influenza virus within the mucus and surfactant films lining the respiratory tract binds to the multiple SA epitopes (SA in rectangles) on the α-gal/SA liposomes landing within these films. 2. The natural anti-Gal antibody binds to the multiple α-gal epitopes (α-Gal in rectangles) on the α-gal/SA liposomes and activates the complement system to produce chemotactic complement cleavage peptides such as C5a, C4a and/or C3a. 3. Monocyte, macrophage and dendritic cells are recruited by the complement cleavage chemotactic peptides toward the α-gal/SA liposomes. 4. The α-gal/SA liposomes with bound influenza virus are internalized by the recruited macrophages and dendritic cells as a result of interaction between the immunocomplexed anti-Gal antibody and Fc receptors (FcR) on the macrophages and dendritic cells. 5. Internalized influenza virus is killed within these macrophages and dendritic cells which further function as antigen presenting cells (APC) that process the influenza virus antigens and transport them to the draining lymph nodes. The next step, not shown in this illustration, is that of macrophages and dendritic cells presenting the processed influenza virus immunogenic peptides to the T cells within the regional lymph nodes in order to elicit protective humoral and cellular immune responses. HA-influenza virus hemagglutinin illustrated as a knobbed protein protruding from the virus envelop and binding SA epitopes; NA-influenza virus neuraminidase illustrated as a filled triangle. The influenza virus is schematically described. Liposome is illustrated as a micelle (one phospholipids layer) because of space limits.

FIG. 3 describes a non-limiting example for the preparation of synthetic or natural α-gal/SA liposomes. The phospholipid phosphatidyl choline or any other natural or synthetic phospholipid is dissolved in an organic solvent such as, but not limited to, methanol. A synthetic or natural α-gal glycolipid, such as, but not limited to Galα1-3Galβ1-4Glcβ1-3Galβ1-4Glc linked to a diacyl lipid is dissolved together with the phosphatidyl choline in methanol at a molar ratio such as, but not limited to 1:10 α-gal glycolipid:phospholipid. A synthetic or natural SA-glycolipid, such as, but not limited to SAα2-6(3)Galβ1-4Glcβ1-3Galβ1-4Glc or SAα2-3Galβ1-4Glcβ1-3Galβ1-4Glc linked to a diacyl lipid is dissolved together with the phosphatidyl choline and the α-gal glycolipid in methanol at a molar ratio such as, but not limited to 1:10 SA-glycolipid:phospholipid. There may be various molar ratios between α-gal glycolipids and SA-glycolipid. The mixture is dried in any other drying device known to those skilled in the art. Subsequently, the dried mixture is sonicated in saline or any other suitable buffer to form synthetic or natural α-gal/SA liposomes comprised of lipid bi-layers of phosphatidyl choline, SA-glycolipid and α-gal glycolipid molecules. These liposomes present multiple SA-epitopes and multiple α-gal epitopes. Synthetic α-gal/SA liposomes may be prepared from any type of lipid, preferably from a phospholipid and from synthetic or natural glycolipids comprised of one or more carbohydrate chains some of which carry α-gal epitopes and the other carry SA-epitopes. The α-gal and SA epitopes may be linked to the lipid by a spacer or directly by a carbohydrate chain. This linking is performed by methods known to those skilled in the art. α-Gal/SA liposomes may be prepared by a similar method using various phospholipid, various α-gal glycolipid(s) and natural SA-glycolipid(s). The α-gal epitopes and SA epitopes may also be linked to the same carbohydrate chain on each glycolipid molecule. α-Gal/SA liposomes may present or contain various molecules in addition to α-gal glycolipids and SA-glycolipids. The figure on the right in this illustration describes an α-gal/SA liposome on which “SA” in rectangles represents SA epitopes and “α-Gal” in rectangles represents α-gal epitopes.

FIG. 4 presents a schematic illustration of the processes occurring following application of α-gal liposomes to injuries in humans. The illustrated α-gal liposomes have α-gal glycolipids, each capped with an α-gal epitope (α-Gal in rectangles). α-Gal glycolipids may have one, two or several branches carrying α-gal epitopes. When α-gal liposomes are applied to an injured tissue the natural anti-Gal antibody binds to α-gal epitopes on the liposomes. This binding of the natural anti-Gal antibody to administered α-gal liposomes activates the complement system. The chemotactic factors C5a and C3a generated as complement cleavage peptides induce rapid recruitment of macrophages to the site of α-gal liposomes. The recruited macrophages interact via their Fc receptors (FcR) with the Fc portion of anti-Gal coating the α-gal liposomes. This interaction activates the macrophages to secrete a wide range of cytokines and growth factors that promote regeneration of the treated injury. Liposome is illustrated as a micelle (one phospholipids layer) because of space limits.

FIG. 5 describes the binding of influenza PR8 virus to α-gal/SA liposomes (FIG. 5A) and to SA liposomes (FIG. 5B). The liposomes were used as solid phase antigen in ELISA wells as 10 μg/ml and the virus was applied at various concentrations as indicated on the X-axis of FIGS. 5A and 5B. FIG. 5 also describes binding of monoclonal anti-Gal antibody M86 to α-gal/SA liposomes (FIG. 5C) and no binding of this antibody to SA liposomes (FIG. 5D) in ELISA wells. The liposomes serving as solid phase antigen in FIGS. 5C and 5D were plated in the ELISA wells at various concentrations, as indicated in the X-axis. Binding of the virus to the liposomes in FIGS. 5A and 5B was measured by using mouse anti-PR8 virus antibody with secondary anti-mouse IgG (Fab)₂ coupled to peroxidase (HRP). Binding of monoclonal anti-Gal antibody to the liposomes in FIGS. 5C and 5D was determined by using anti-mouse IgM-HRP as secondary antibody.

FIG. 6 demonstrates in α1,3galactosyltransferase knockout mice (GT-KO mice) the ability of α-gal/SA liposomes and of SA liposomes to inhibit progression of influenza virus infection (details in Example 2 below). Anti-Gal producing GT-KO mice received by intranasal inoculation a sub-lethal dose of A/Puerto Rico/8/34-H1N1 influenza virus (PR8 virus). Subsequently, the mice are subjected to inhalation of α-gal/SA liposomes, SA liposomes or saline and monitored for body weight changes. The inhalation was performed 3 times on Days 0-3, twice on Days 4 and 5 and once on Days 6 and 7. FIGS. 6A and 6B—mice infected with PR8 virus and inhaled saline () (n=5). FIG. 6A—mice inhaling α-gal/SA liposomes post PR8 infection (◯) (n=5). FIG. 6B—mice inhaling SA liposomes post PR8 infection (◯) (n=5). Note that inhaling α-gal/SA liposomes or SA liposomes decreases the extent of weight loss in the mice infected with PR8 (i.e. lessens the infection) and induces an earlier recovery than in the absence of these liposomes.

FIG. 7 describes the results for binding of IgG antibodies from sera of 6 GT-KO mice (), or 6 wild type (WT) mice (◯) to liposomes presenting α-gal epitopes, used as solid phase antigen in ELISA wells. Both mouse strains were immunized 3 times in one week intervals with 50 mg pig kidney membranes (PKM) homogenate. Note that binding is observed only in GT-KO mouse sera because of anti-Gal binding to α-gal epitopes on the liposomes. WT mice produce no anti-Gal antibody since they synthesize the α-gal epitope on their cells as a self-antigen and thus, their sera display no IgG binding to the α-gal liposomes.

FIG. 8 provides exemplary data demonstrating in vivo recruitment of macrophages into polyvinyl alcohol (PVA) sponge containing α-gal liposomes. The sponges containing 10 mg α-gal liposomes in suspension were implanted subcutaneously in GT-KO mice for 3, 6, or 9 days, then removed. The cells infiltrating within the sponge were obtained by repeated squeezing of the sponge in 1 ml phosphate buffered saline (PBS). FIG. 8A—Quantification of macrophages migrating into PVA sponge discs containing 10 mg α-gal liposomes or saline, at different time points. The PVA sponge discs were implanted subcutaneously in GT-KO mice producing anti-Gal (KO mice), or in wild type (WT) mice that were immunized with pig kidney membranes (PKM), similarly to KO mice, but which lack the anti-Gal antibody. Data presented as mean+standard deviation of 5 mice/group. FIG. 8B—Immunostaining of cells recruited by the anti-Gal/α-gal liposomes interaction. Infiltrating cells were retrieved from PVA sponge discs containing 10 mg α-gal liposomes, 6 days post-subcutaneous implantation. The cells were subjected to flow cytometry analysis of various surface population markers by evaluating binding of the corresponding antibodies. Note that the large majority of infiltrating cells are macrophages characterized by expression of CD11b and CD14, whereas no significant infiltration of T cells, or B cells is observed. Similar staining patterns were observed in cell populations obtained 3 and 9 days post-implantation (representative data of 5 mice).

FIG. 9 describes the analysis by ELISPOT assay of IFN-γ secretion levels in anti-Gal producing GT-KO mice immunized twice in 1 week interval with 1 μg inactivated PR8_{αgal} virus (PR8 virus presenting multiple α-gal epitopes; mice #1-6) or with PR8 influenza virus (PR8 virus lacking α-gal epitopes; mice #7-12). Lymphocytes from the mice were obtained 14 days after the second immunization and incubated with dendritic cells of the dendritic cell line DC2.4 and subjected to ELISPOT (hatched columns). Lymphocytes incubated dendritic cells that were not pulsed by PR8 virus (open columns). The data are presented as means+standard deviation of the results for triplicate wells.

FIG. 10 describes results for production of anti-PR8 antibodies in mice immunized twice with 1 μg inactivated PR8_{αgal} () or with inactivated PR8 (∘) (as in FIG. 9) and measured by ELISA with PR8 virus as a solid-phase antigen. (A) Anti-PR8 IgG response in anti-Gal producing GT-KO mice. (B) Anti-PR8 IgG response in WT mice. (C) Anti-PR8 IgA response in anti-Gal producing GT-KO mice (n=6 per group). The two GT-KO mice in panels A and C with the lowest levels of response () are mice no. 5 and 6 in FIG. 9 above.

FIG. 11 describes the survival rates of anti-Gal producing GT-KO mice immunized twice with inactivated PR8 virus (∘) or with inactivated PR8_{αgal} virus () and receiving intranasal challenge with live PR8 (immunization as in FIG. 9). The immunized mice were challenged intranasally with 2,000 plaque forming units (PFU) of live PR8 virus in 50 μl aliquots (n=25/group). Survival data are presented as percentages of live mice at various time points post-challenge. The survival data for day 30 were similar to those for day 15 post-challenge.

### DEFINITIONS

To facilitate an understanding of the present invention, a number of terms and phrases are defined below:

Carbohydrate abbreviations: Fuc-fucose; Gal-galactose; GalNAc-N-acetylgalactosamine; Glc-glucose; GlcNAc-N-acetylglucosamine; Man-mannose; SA-sialic acid.

The term “lipid” as used herein, refers to any molecule from a group of naturally occurring or synthetic molecules that include: fats, waxes, sterols, fat soluble vitamins, monoglycerides, diglycerides, triglycerides and phospholipids.

The term “α-gal epitope” as used herein, refers to any molecule or part of a molecule, with a terminal structure comprising Galα1-3Galβ1-4GlcNAc-R, Galα1-3Galβ1-3GlcNAc-R, or any carbohydrate chain with terminal Galα1-3Gal at the non-reducing end, i.e., galactosyl linked α1-3 to a galactosyl, or any molecule with terminal α-galactosyl unit at the non-reducing end and capable of binding the anti-Gal antibody. The α-gal epitope may be of natural source or of synthetic source.

The term “glycolipid” as used herein, refers to any molecule with at least one carbohydrate chain linked to a ceramide, or a fatty acid chain, or any other lipid. Alternatively, a glycolipid maybe referred to as a glycosphingolipid. Glycolipids may be of natural or synthetic origin and may include a linker between a carbohydrate epitope and a ceramide, or a fatty acid chain, or any other lipid.

The term “α-gal glycolipid” as used herein, refers to any glycolipid that has at least one α-gal epitope at its non-reducing end of the carbohydrate chain or linked to any other linker and may be of natural or synthetic origin.

The term “α-gal liposomes” as used herein, refers to any liposomes comprised of natural or synthetic phospholipids, or other lipids, which is also comprised of hydrocarbon base, or any other base which contains natural or synthetic α-gal epitopes or α-gal epitopes in natural or synthetic α-gal glycolipids, or α-gal proteins, or α-gal proteoglycans, or α-gal polymers, or any other molecule carrying α-gal epitopes. α-Gal liposomes may or may not have also cholesterol in their membrane. The liposome can be of any size provided that it has one or more lipid bilayer and the materials comprising them can be of natural or synthetic origin. The term “synthetic α-gal liposomes” as used herein, refers to liposomes comprised of natural or synthetic lipids, such as but not limited to phosphatidyl choline, phosphatidyl ethanolamine, phosphatidyl serine and synthetic α-gal glycolipids or any other synthetic molecules that bind the natural anti-Gal antibody.

The term “micelle” is defined here as a spherical structure comprising lipids, including but not limited to phospholipids and glycolipids in which the hydrophobic tails of the molecules are facing each other within the inner space of the sphere and the hydrophilic part faces the aqueous surrounding.

The term “α-gal nanoparticles” as used herein, refers to an α-gal liposomes with a submicroscopic size, comprised of natural or synthetic materials and present natural or synthetic α-gal epitopes. α-Gal epitopes may be part of α-gal glycolipids, α-gal glycoproteins, α-gal proteoglycans, synthetic molecules carrying α-gal epitopes, or α-gal polymers. The term “synthetic α-gal nanoparticles” as used herein, refers to nanoparticles comprised of natural or synthetic lipids, such as, but not limited to phosphatidyl choline, phosphatidyl ethanolamine, phosphatidyl serine and synthetic α-gal glycolipids or any other synthetic molecules that bind the natural anti-Gal antibody.

The term SA used herein refers to sialic acid. The sialic acid may be N-glycolyl neuraminic acid (Neu5Gc), or preferably N-acetyl neuraminic acid (Neu5Ac).

The term “SA epitope” as used herein, refers to any molecule or part of a molecule, with a terminal structure at a non-reducing end, including but not limited to sialic acid (SA) linked α2-6 to a penultimate galactose as SAα2-6Gal-R, sialic acid linked α2-3 to galactose as SAα2-3Gal-R, sialic acid linked α2-8 to sialic acid as SAα2-8SA-R, or SAα2-6Galβ1-4GlcNAc-R, SAα2-3Galβ1-4GlcNAc-R, SAα2-6GalNAc-R and/or SAα2-3GalNAc-R or any carbohydrate portion at a non-reducing end of a ganglioside that includes terminal sialic acid (SA) at the non-reducing end, or any molecule with terminal SA unit, where R is any natural or synthetic carbohydrate linked to glycolipid, glycoprotein, proteoglycan or polymer, or any other natural or synthetic linker, or both synthetic and natural linker that links the sialic acid epitope to a glycolipid, glycoprotein, proteoglycan, polymer or any other molecule. The SA epitope may be of natural source or of synthetic source. SA epitopes and α-gal epitopes may be linked to separate glycolipids, glycoproteins, proteoglycans or polymers, or to the same glycolipid, glycoprotein, proteoglycan or polymer.

The term SA-glycolipid as used herein, refers to any glycolipid that has at least one SA-epitope on its non-reducing end of the carbohydrate chain or linked to any other linker and may be of natural or synthetic origin. SA-glycolipids are also referred to as gangliosides.

The term “α-gal/SA liposomes” as used herein, refers to α-gal liposomes that also comprise of SA-glycolipids or SA epitopes linked to glycoprotein, proteoglycan or polymer, or any other natural or synthetic linker or both synthetic and natural linker that links the sialic acid epitope to a glycolipid, glycoprotein, proteoglycan or polymer. α-gal/SA liposomes present on their surface multiple α-gal epitopes and multiple SA-epitopes of natural or synthetic origin.

As used herein, the term “purified” refers to molecules (polynucleotides, or polypeptides, or glycolipids) that are removed from their natural environment, isolated or separated. “Substantially purified” molecules are at least 50% free, preferably at least 75% free, more preferably at least 90% and most preferably at least 95% free from other components with which they are naturally associated.

The terms “α1,3-galactosyltransferase,” “α-1,3-galactosyltransferase,” “α1,3GT,” “α-galactosyltransferase” and “GGTA1,” as used herein refer to any enzyme capable of synthesizing α-gal epitopes. The enzyme is expressed in nonprimate mammals but not in humans, apes and Old World monkeys. The carbohydrate structure produced by the enzyme is immunogenic in man and most healthy people have high titer natural anti α-gal antibodies, also referred to as “anti-Gal” antibodies. In some embodiments, the term “α1,3GT” refers to a common marmoset gene (e.g., Callithrix jacchus—GENBANK Accession No. S71333) and its gene product, as well as its functional mammalian counterparts (e.g., other New World monkeys, prosimians and non-primate mammals, but not Old World monkeys, apes and humans). In other embodiments, the term “α1,3GT” refers to mouse α1,3GT (e.g., Mus musculus—nucleotides 445 to 1560 of GENBANK Accession No. NM_010283), bovine α1,3GT (e.g., Bos taurus—GENBANK Accession No. NM_177511), feline α1,3GT (e.g., Felis catus—GENBANK Accession No. NM_001009308), ovine α1,3GT (e.g., Ovis aries—GENBANK Accession No. NM_001009764), rat α1,3GT (e.g., Rattus norvegicus—GENBANK Accession No. NM_145674) and porcine α1,3GT (e.g., Sus scrofa—GENBANK Accession No. NM_213810).

The term “anti-Gal binding epitope”, as used herein, refers to any molecule or part of molecule that is capable of binding in vivo the natural anti-Gal antibody.

The term “anti-Gal antibody”, as used herein, refers to a natural antibody present in large amounts in humans, apes and Old World monkeys, or in other vertebrate lacking α-gal epitopes, such as birds, and which binds to antigens carrying α-gal epitopes, molecules and peptides mimetic to α-gal epitopes and other carbohydrates that mimic α-gal epitopes structure or are part of this structure.

The term “isolated” as used herein, refers to any composition or mixture that has undergone a laboratory purification procedure including, but not limited to, extraction, centrifugation and chromatographic separation (e.g., thin layer chromatography or high performance liquid chromatography). Usually such purification procedures provide an isolated composition or mixture based upon physical, chemical, or electrical potential properties. Depending upon the choice of procedure an isolated composition or mixture may contain other compositions, compounds or mixtures having similar chemical properties.

The term “control” refers to subjects or samples which provide a basis for comparison for experimental subjects or samples. For instance, the use of control subjects or samples permits determinations to be madc regarding the efficacy of experimental procedures. In some embodiments, the term “control subject” refers to animals, which receive a mock treatment (e.g., saline or inactivated influenza virus lacking α-gal epitopes).

The terms “patient” and “subject” refer to a human, a mammal, a bird, or an animal that is a candidate for receiving medical treatment.

The term “cell migration” refers to the movement of cells (e.g., macrophages, dendritic cells etc.) to the injured or treated tissue.

As used herein, “α-gal/SA liposomes suspension” include, but are not limited to conventional suspensions of α-gal/SA liposomes in a fluid aqueous vehicle such as, but not limited to, saline (physiological sodium chloride solutions), phosphate buffered saline, or any other fluid or gel. Suitable additives or auxiliary substances are isotonic solutions, such as physiological sodium chloride solutions or sodium alginate, demineralized water and stabilizers. The α-gal/SA liposomes suspension may be delivered as inhaled aerosol.

### GENERAL DESCRIPTION OF THE INVENTION

### General

The present invention relates to the fields of treatment of microbial respiratory infections and delivery of microbial vaccines in general and influenza virus infections and influenza virus vaccines in particular. The present invention provides compositions and methods for preventing or slowing growth of influenza virus in symptomatic patients and for induction of a potent immune response by targeting influenza virus antigens or other microbial antigen of interest to antigen presenting cells (APC) of a treated patient. As described herein, this targeting is achieved by harnessing the immunologic potential of the natural anti-Gal antibody, which is the most abundant natural antibody in humans constituting ˜1% of immunoglobulins. This antibody interacts specifically with the carbohydrate epitope called the α-gal epitope with the structure Galα1-3Galβ1-4GlcNAc-R, or Galα1-3Galβ1-3GlcNAc-R, or Galα1-3Galα1-4Glc-R, or Galα1-3Galβ1-3Glc-R (Galili, supra Immunology 2013). In addition, this invention exploits the requirement for influenza virus to bind to sialic acid epitopes (SA epitopes) in order to infect cells whereas other respiratory viruses use a variety of similar or different epitopes as “docking” receptors on cells they infect.

### I. Influenza Virus Infection and Current Treatments

Influenza, commonly known as “the flu”, is an infectious disease caused by the influenza (flu) virus (“Influenza (Seasonal) Fact sheet No 211”. who. int. March 2014). Symptoms can be mild to severe. The symptoms of influenza usually are observed within two days after infection by the influenza virus and include high fever, runny nose, sore throat, muscle pains, headache and coughing. The disease may be exacerbated because of complications including viral pneumonia, secondary bacterial pneumonia, sinus infections, and worsening of previous health problems such as asthma or heart failure. The virus is spread through the air from coughs, sneezes, or talks and the spread is most effective in closed places such as public transportation, movie theaters, malls and other public gathering places Influenza spreads around the world in a yearly seasonal outbreak, resulting in about three to five million cases of severe illness and about 250,000 to 500,000 deaths. Death occurs mostly in the very young, the old and those with other health problems.

The vaccines against influenza virus has an efficacy of ˜75% in young populations and no more than 50% in elderly populations. Once a person is infected with the virus, treatment may include two classes of antiviral drugs used against influenza which are neuraminidase inhibitors (Oseltamivir© and Zanamivir©) and M2 protein inhibitors (adamantane derivatives that inhibits the M2 viral ion channels). The efficacy of these treatments is limited, thus individuals infected with influenza virus and who become symptomatic may benefit from additional treatments that can prevent further infection by the virus and induce effective destruction of the infectious virus. The present invention teaches a novel method for achieving these objectives by inhalation of α-gal/sialic acid liposomes (referred to in this application as α-gal/SA liposomes).

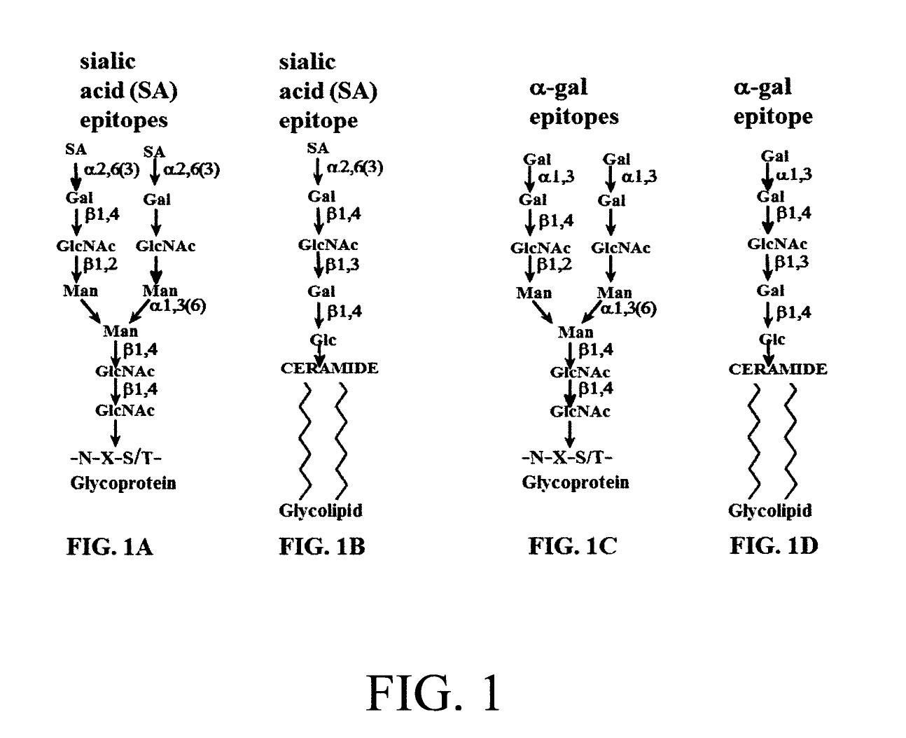

The influenza virus penetrating into the respiratory tract attaches itself to the epithelium lining the respiratory tract by binding to a carbohydrate called sialic acid (SA) on cell surface glycoproteins, glycolipids (FIG. 1) and proteoglycans (collectively known as glycoconjugates). The binding of influenza virus to SA on cell surface glycoconjugates is mediated by the main viral envelope glycoprotein called hemagglutinin (HA). The viral hemagglutinin is a glycoprotein that carries SA when it is produced in the host cells. The viral SA on HA is removed by a second envelope protein called neuraminidase (NA) which cleaves the SA units on the virus and on cell surface glycoconjugates, thereby it releases the virus from the cell membrane of the infected cells. The removal of SA from the HA of influenza virus further prevents binding of HA on one virus to SA on HA of another virus and thus prevents generating aggregates of the virus. As indicated above, the ability of neuraminidase inhibitors such as Oseltamivir (Tamiinfluenza®), Zanamivir (Relenza®) or Peramivir (Rapivab®) to prevent progression of influenza virus infection is suboptimal, thus patients infected with influenza virus may benefit from the use of additional treatments that can slow virus growth. The efficacy of these neuraminidase inhibitor drugs in inducing an effective slowing of the influenza virus infection is still controversial since some clinical studies reported no beneficial effects whereas others reported some clinical effects. The process of binding the influenza virus to SA on the cell surface membrane of cells of the epithelium which lines the respiratory tract is a stage that can be subjected to intervention for slowing or preventing the influenza virus growth cycle. Such intervention can delay and possibly prevent viral infection and spread in the respiratory epithelium. This invention teaches how to prevent binding of influenza virus to SA of the respiratory epithelium, then to induce destruction of the virus and to rapidly convert the infecting virus into an effective in situ vaccine by inhalation of α-gal/SA liposomes and thus exploitation of the natural anti-Gal antibody for achieving these objectives.

### II. Structure of α-Gal/SA Liposomes

The present invention is related to the field of preventing infections of the respiratory tract by respiratory viruses and bacteria. In particular, the present invention provides compositions and methods for preventing infection of respiratory epithelium by inducing binding of infective influenza virus to SA epitopes on α-gal/SA liposomes thereby preventing the virus from infecting the respiratory epithelium. Liposomes that deliver various drugs by inhalation have been studied in humans. For example liposomes delivering amikacin to the lungs have been evaluated in patients with cystic fibrosis (Okusanya et al. Antimicrob Agents Chemother. 58: 5005, 2014) and liposomes delivering insulin via the lungs were studied in diabetic patients (review by Siekmeier and Scheuch J Physiol Pharmacol. 59: 81, 2008).

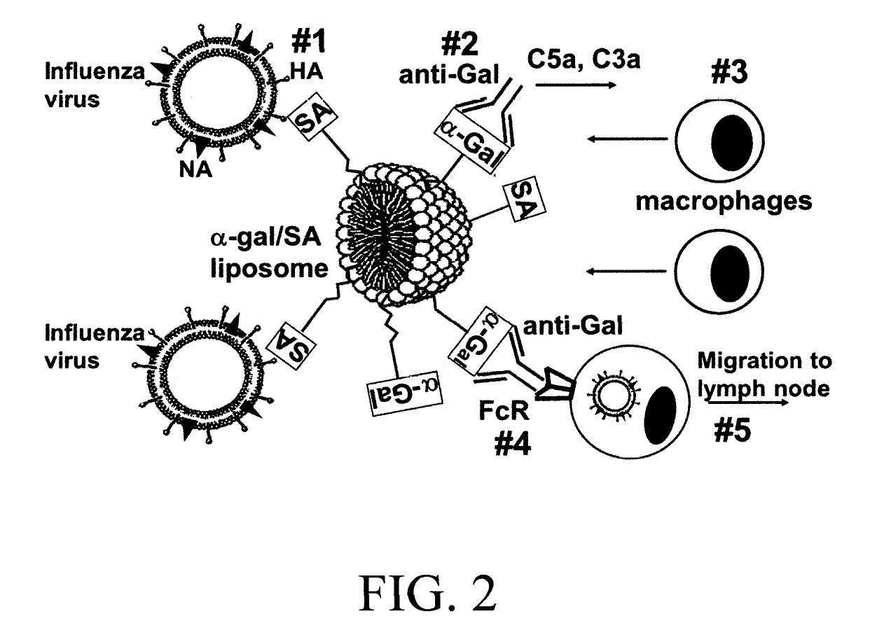

This invention teaches the preparation and clinical use of α-gal/SA liposomes which are liposomes that present both multiple α-gal epitopes and multiple SA epitopes. This type of liposomes presenting both α-gal epitopes and SA epitopes is novel and has not been previously reported. This invention teaches how infecting influenza virus binds to SA epitopes on inhaled α-gal/SA liposomes. The invention further teaches how to induce rapid recruitment of macrophages to the surface of the respiratory epithelium by the interaction of the α-gal epitopes on the α-gal/SA liposomes with the natural anti-Gal antibody and the activation of the complement system as result of this interaction (FIG. 2). The invention also teaches how complement cleavage chemotactic peptides produced as a result of complement activation induce rapid recruitment of macrophage and how the natural anti-Gal antibody bound to α-gal/SA liposomes induces effective internalization into macrophages of the influenza virus bound to α-gal/SA liposomes. This internalization (i.e. uptake) of the virus bound to the α-gal/SA liposomes occurs following interaction between the Fc portion of anti-Gal coating these liposomes and Fc receptors (FcR) on the recruited macrophages, further resulting in the destruction of the internalized virus by the macrophages (FIG. 2). Moreover, this invention teaches how the influenza virus internalized by the macrophages and dendritic cells is converted by these cells into an effective vaccine that induces rapid protective immune response against the infecting virus. The recruited macrophages and dendritic cells function as antigen presenting cells (APC) that process the viral antigens into immunogenic peptides and transport these peptides to the regional lymph nodes. The virus immunogenic peptides are further presented on macrophages and dendritic cells in association with Class I and Class II MHC molecules for eliciting an effective protective anti-viral humoral and cellular immune responses. Such an immune response enables the effective termination of the influenza virus infection shortly after it was initiated, thereby inducing earlier recovery from the infection in comparison to physiologic recovery, preventing complications of this disease and decreasing the morbidity and mortality following influenza virus infection.

In some embodiments, the α-gal epitope on the α-gal/SA liposomes is selected from the group consisting of but not limited to Galα1-3Gal-R, Galα1-2Gal-R, Galα1-6Gal-R and Galα1-6Glc-R. The α-gal epitopes on the α-gal/SA liposomes further may be prepared from oligosaccharides available from Dextra (Reading, UK), but are not limited to: i) Galα1-3Gal glycolipids: α1-3 galactobiose (cat. # G203); linear B-2 trisaccharide (cat. # GN334); and Galili pentasaccharide (cat. # L537). Various other glycoconjugates with α-gal epitopes available from Dextra include for instance: Galα1-3Galβ1-4Glc-BSA (cat. # NGP0330); Galα1-3Galβ1-4(3-deoxyGlcNAc)-HAS (cat. # NGP2335); Galα1-3Galβ1-4GlcNAcβ1-HDPE (cat. # NGL0334); and Galα1-3Gal-BSA (cat. # NGP0203) all which may be linked to a lipid or to other materials that form α-gal/SA liposomes. Another non-limiting example is the Elicityl (Grenoble, France) Galα1-3Gal series of carbohydrate chains of various sizes carrying α-gal epitopes (called “Galili series”). All these α-gal epitopes may be linked by a carbohydrate chain or by any linker to a lipid or to other materials that form liposomes. An additional non-limiting example is a synthetic glycolipid with an α-gal epitope called “FSL-Galili” produced by KODE Biothec (Auckland, NZ) and distributed by KODE Biothec and by Sigma-Aldrich Inc. as catalogue number (cat. # F9432). The α-gal epitope on glycolipids, or glycoproteins or proteoglycan may be of natural sources, such as, but not limited to rabbit red cell membranes, bovine or porcine red cell membranes. The α-gal epitope on glycolipids, or glycoproteins or proteoglycans or polymers that may be used for preparation of α-gal/SA liposomes may also be of synthetic origin produced by any chemical, biochemical or enzymatic methods known to those skilled in the art.

The sialic acid epitopes (SA epitopes) on the α-gal/SA liposomes include oligosaccharides with terminal SA at the non-reducing end and linked to ceramide or to proteins that may or may not be linked to a lipid tail. Such oligosaccharides with SA at the non-reducing end that may be linked to a lipid are available from Dextra (Reading, UK), but are not limited to: i) 3′-Sialyl-N-acetyllactosamine (3′-SLN)-(cat. # SLN302), 3′-Sialyllactose (3′-SL)-(cat. # SL302), 6′-Sialyl-N-acetyllactosamine (6′-SLN)-(cat. # SLN306), 6′-Sialyllactose (6′-SL)-(cat. # SL306). Another non-limiting example is the Elicityl (Grenoble, France) series of carbohydrate chains of various sizes carrying terminal SA at the non-reducing end and having or lacking a linker all which may be linked to a lipid or to other materials that form liposomes. The Elicityl produced carbohydrate chains carrying SA include, but are not limited to Neu5Acα2-3Galβ1-3GlcNAcβ1-3Galβ1-4Glc (cat. # GLY081), Neu5Acα2-3Galβ1-4GlcNAcβ1-3Galβ1-4Glc (cat. # GLY083), or Neu5Acα2-3Galβ1-3GlcNAcβ1-3Gal (cat. # GLY080). In addition, natural or synthetic glycoproteins such as but not limited to human or other mammalian α2-acid glycoprotein, and fetuin, as well as natural or synthetic glycolipids which carry sialic acid at the non-reducing end of the carbohydrate chain may serve as suitable sources for preparation of α-gal/SA liposomes. An additional non-limiting example is a synthetic glycolipids with terminal sialic acid produced by KODE Biothech (Auckland, New Zealand) and distributed by KODE Biothech and by Sigma Aldrich Inc.

Several non-limiting examples of additional macromolecules that carry α-gal epitopes and thus may be used for preparation of α-gal/SA liposomes include but are not limited to: mouse laminin with 50-70 α-gal epitopes (Galili, Springer Seminars in Immunopathology, 15:155, 1993), multiple synthetic α-gal epitopes linked to BSA (Stone et al., Transplantation, 83:201, 2007), GAS914 produced by Novartis (Zhong et al., Transplantation 75:10, 2003), the α-gal polyethylene glycol conjugate TPC (Schirmer et al., Xenotransplantation, 11: 436, 2004), α-gal epitope mimicking peptides linked to a macromolecule backbone (Sandrin et al. Glycocon J 14: 97, 1997) and rabbit α-gal glycolipids from red cell membranes that are isolated (Galili et al. supra J Immunol 2007). Mixing these natural or synthetic α-gal epitope carrying molecules with molecules carrying SA epitopes and with phospholipids can be used for preparation of α-gal/SA liposomes by methods known to those skilled in the art.

In addition, chloroform:methanol extracts or other organic solution extracts bovine red cells membranes include a mixture of glycolipids with α-gal epitopes (α-gal glycolipids) (Galili et al. Proc Natl Acad Sci USA 84: 1369, 1987), glycolipids carrying SA (gangliosides) (Chien et al. J Biol Chem 253: 4031, 1978; Uemura et al. J Biochem 83: 463, 1978), glycolipids carrying both α-gal epitopes and SA (Watanabe et al. J Biol Chem 254: 3221,1979), phospholipids and with or without cholesterol are suitable for preparation of α-gal/SA liposomes. The α-gal/SA liposomes produced from biological sources such as various red cell membranes or other types of tissues, may include α-gal glycolipids, gangliosides, glycolipids carrying both α-gal epitopes and SA epitopes, phospholipids. These α-gal/SA liposomes may or may not include also cholesterol and other glycolipids, glycoproteins, proteoglycans or other polymers are suitable for preparation of α-gal/SA liposomes, provided that the other molecules do not interfere with the interaction of influenza virus with SA epitopes and interaction of the anti-Gal antibody with α-gal epitopes.

In some preferred embodiments, the α-gal epitopes and the SA epitopes used for preparation of α-gal/SA liposomes are parts of molecules selected from the group consisting of glycolipids (e.g., α-gal epitopes or SA epitopes on carbohydrate chain that is linked to ceramide), glycoproteins (e.g., α-gal albumin and SA albumin), proteoglycans, glycopolymers (e.g., α-gal polyethylene glycol mixed with SA on polyethylene glycol or polyethylene glycol carrying both SA and α-gal epitopes) and any other natural or synthetic spacer. In some particularly preferred embodiments, α-gal/SA liposomes are liposomes that have on their surface α-gal epitopes that are capable of binding the anti-Gal antibody and SA epitopes that are capable of binding influenza virus via the hemagglutinin (HA) protein on the virions. Also provided are methods in which the preparation further comprises anti-Gal antibodies bound to the α-gal/SA liposomes.

In some embodiments, the α-gal glycolipids and gangliosides (SA carrying glycolipids) preparations comprising α-gal/SA liposomes are derived from a source selected from the group consisting of rabbit red blood cells, bovine red blood cells, and other non-primate mammalian cells. In another embodiment the α-gal glycolipids and gangliosides preparations comprising synthetic α-gal liposomes are derived from synthetic α-gal glycolipids, synthetic gangliosides and phospholipids, or from a mixture of natural and synthetic α-gal glycolipids, synthetic gangliosides and phospholipids, or from such natural compound. α-gal/SA liposomes may or may not include cholesterol in their lipid bi-layer or in their micelle structure. In addition, the present invention provides methods, comprising: providing; a subject having endogenous anti-Gal antibody and infecting influenza virus and a preparation comprising suspension of liposomes presenting both multiple α-gal epitopes and SA epitopes and applying the preparation to influenza virus infected respiratory tract by inhalation of aerosol containing said liposomes. In view of studies on affinity of influenza virus to various glycolipids with terminal non-reducing sialic acid (Rogers and Paulson Virology 127: 361, 1983; Suzuki et al. J Biol Chem 261: 17057, 1986), in some embodiments, the terminal sialic acid (SA) is selected from the group consisting of but not limited to SAα2-6Gal-R, SAα2-3Gal-R, SAα2-6GalNAc-R and/or SAα2-3GalNAc-R where R represents the rest of the glycolipid molecule.

In some preferred embodiments, the α-gal epitope is part of a natural or synthetic molecule selected from the group consisting of a glycolipid such as but no limited to α-gal epitope linked to ceramide, a glycoprotein such as but not limited to α-gal albumin, proteoglycan and a glycopolymer such as but not limited to α-gal polyethylene glycol. The SA epitope in the α-gal/SA liposomes is part of a molecule selected from the group consisting of a glycolipid such as but not limited to SA epitope linked via a carbohydrate chain or via a spacer to a ceramide or to any other lipid “tail”, a glycoprotein such as but not limited to α-gal albumin and SA-albumin, proteoglycan and a glycopolymer such as but not limited to α-gal polyethylene glycol and SA-polyethylene glycol and/or polyethylene glycol on which some of the branches carry α-gal epitopes and other branches carry SA-epitopes. Also provided are methods in which the preparation of further compositions comprises anti-Gal antibodies bound to the α-gal/SA liposomes.

In some embodiments, the preparation is selected from the group consisting of biodegradable material such as collagen, alginate or cellulose, biological matrices, hydrocolloid, hydrogel, phospholipids and other biodegradable materials that can be aerosolized and multiple SA-epitopes and α-gal epitopes can be linked to said biodegradable materials. Such biodegradable materials carrying both α-gal epitopes and SA-epitopes can bind influenza virus by SA/hemagglutinin interaction and further bind the anti-Gal antibody via the α-gal epitopes.

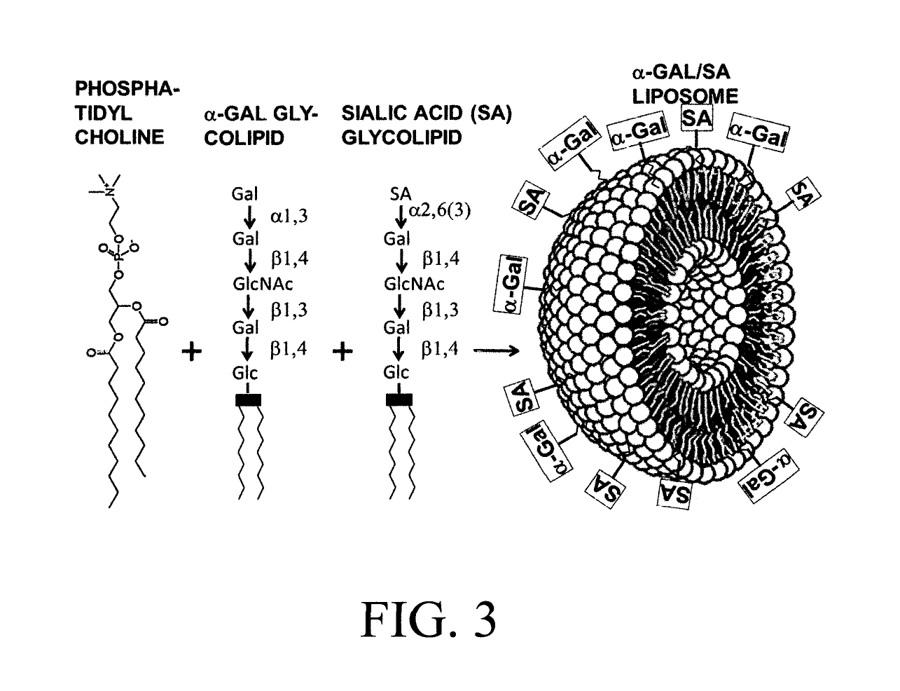

A non-limiting example for the preparation of α-gal/SA liposomes is illustrated in FIG. 3 where natural or synthetic α-gal glycolipids are mixed with natural or synthetic glycolipids with terminal sialic acid (called “SA-glycolipids” or “gangliosides”) and with phospholipids such as, but not limited to phosphatidyl choline. All these molecules are dissolved in an organic solvent such as, but not limited to chloroform:methanol and dried in a rotary evaporator or by any other method known to those skilled in the art. Subsequently, the dried mixture is sonicated in saline or other physiologic buffer, to generate α-gal/SA liposomes illustrated in the right portion of FIG. 3. The α-gal/SA liposomes are further sonicated to reduce their size to a size lower than 300 nm so that they can be filtered through pores of sterilizing filters which remove any bacteria accidentally present in the suspension and which are known to those skilled in the art.

III. Inhaled α-Gal/SA Liposomes within the Respiratory Tract

The α-gal epitopes and the SA epitopes on α-gal/SA liposomes have two different functions. The interaction of SA epitopes on α-gal/SA liposomes with hemagglutinin protein molecules on the envelope of the influenza virus prevents the binding of influenza virus to the SA epitopes on respiratory tract epithelium glycoproteins, glycolipids and proteoglycans and thus prevents the penetration of the virus into the cells of the respiratory epithelium. By this function, the SA epitopes on the α-gal/SA liposomes act as a decoy preventing virus binding to the respiratory epithelium cells. The α-gal epitopes on inhaled α-gal/SA liposomes bind the natural anti-Gal antibody which is the most abundant natural antibody in humans constituting ˜1% of immunoglobulins (Galili et al. J Exp Med 1984, supra; Galili et al. 162: 573, 1985). This antigen/antibody interaction activates the complement system which generates chemotactic complement cleavage peptides that induce recruitment of leukocytes, primarily monocytes, macrophages and dendritic cells (Galili et al. J Immunol, supra 2007; Galili et al. Burns 36:239, 2010). The recruited cells reach the α-gal/SA liposomes, bind the Fc “tail” of anti-Gal coating these liposomes and are induced to internalize the anti-Gal coated α-gal/SA liposomes and destroy the influenza virus bound to these liposomes. These recruited macrophages and dendritic cells further function as antigen presenting cells (APC) that process the internalized virus to generate immunogenic peptides. These APC further transport processed virus immunogenic peptides to the regional lymph nodes where these APC present the processed immunogenic peptides in association with MHC class I and class II cell surface molecules for the activation of influenza virus specific T lymphocytes. These activated T cells further activate the immune system to mount a protective humoral and cellular immune response against the infecting influenza virus (Abdel-motal J Virol 81: 9131, 2007).

In some preferred embodiments, the inhaled α-gal/SA liposomes land in the mucus lining the respiratory epithelium and activate the complement system as a result of the natural anti-Gal antibody interacting with α-gal epitopes presented on these liposomes. In some embodiments, complement activation comprises production of C5a, C4a and/or C3a complement cleavage chemotactic peptides. In some preferred embodiments, the inhaled α-gal/SA liposomes are under conditions such that one or more of the followings take place (partly illustrated in FIG. 2): monocyte, macrophage and dendritic cell are recruited by these newly generated complement cleavage chemotactic peptides and migrate toward the α-gal/SA liposomes that land in the mucus lining the respiratory epithelium; influenza virus binds to the SA-epitopes on the α-gal/SA liposomes; the α-gal/SA liposomes with bound influenza virus are taken up (i.e. internalized) by the macrophages and dendritic cells as a result of interaction between the immunocomplexed anti-Gal antibody and Fc receptors (FcR) on the macrophages and dendritic cells; influenza virus is killed within these macrophages and dendritic cells; macrophages and dendritic cells process the influenza virus antigens into peptides and transport them to the regional lymph nodes. The macrophages and dendritic cells further present the processed influenza virus antigenic peptides to the T cells within the regional lymph nodes and thus elicit rapid protective humoral and cellular immune responses. In some embodiments, the subject treated by inhalation of α-gal/SA liposomes is selected from the group consisting of a human, an ape, an Old World monkey, and a bird.

In some embodiments, the glycolipid preparation is derived from a source selected from the group consisting of rabbit red blood cells, bovine red blood cells, human red cells and other mammalian cells or bird cells and are comprised of glycolipids with α-gal epitopes (also called α-gal glycolipids) or glycolipids with sialic acid (SA) epitopes (also called SA-glycolipids or gangliosides), or both. In some embodiments the glycolipids with α-gal epitopes and glycolipids with SA-epitopes comprise liposomes that may also comprise natural or synthetic lipids including but not limited to phospholipids and triglycerides. Such liposomes may or may not also comprise cholesterol. Also provided are methods in which the liposomes preparation further comprises an antibiotic or vitamins. Moreover, in some particularly preferred embodiments, the applied liposomes comprises of α-gal glycolipids and SA glycolipids are delivered by inhalation, or by any other application method known to those skilled in the art. In yet another embodiment the anti-Gal antibody is bound to α-gal/SA liposomes already in the suspension that is to be inhaled as aerosol released by a nebulizing device to the airways of the treated patient or by any other inhalation device known to those skilled in the art. In some embodiments the inhaled aerosol droplets contain molecules or macromolecules that carry both α-gal epitopes and SA epitopes and referred to as α-gal/SA molecules. Such α-gal/SA molecules carrying both α-gal epitopes and SA epitopes, bind influenza virus via SA/hemagglutinin interaction and bind the natural anti-Gal antibody which interacts with α-gal epitopes on these molecules.

In some preferred embodiments, the inhalation of these α-gal/SA molecules is under conditions such that complement activation in the treated respiratory tract is enhanced as a result of anti-Gal binding to these α-gal/SA molecules. In some embodiments, the complement activation comprises production of C5a, C4a and C3a. In some preferred embodiments, the inhaled α-gal/SA molecules are under conditions such that one or more of the following take place: monocyte and macrophage recruitment toward the α-gal/SA molecules that land in the mucus lining the respiratory epithelium is enhanced; influenza virus binds to the SA-epitopes on the α-gal/SA molecules; the α-gal/SA molecules with bound influenza virus are taken up by the macrophages and dendritic cells as a result of interaction between the Fc “tail” of the anti-Gal antibody immunocomplexed to the α-gal/SA molecules and FcR on these recruited cells; the internalized virus is killed within these cells; macrophages and dendritic cells further process the influenza virus antigens and transport them to the regional lymph nodes. The macrophages and dendritic cells present the processed influenza virus immunogenic peptides to T cells within the lymph nodes in order to elicit protective humoral and cellular immune responses. In some embodiments, the subject is selected from the group consisting of a human, an ape, an Old World monkey, and a bird.

In another embodiment, the present invention contemplates treatment of respiratory diseases by the inhalation of liposomes that comprise also of carbohydrate antigens or other antigens which bind antibodies circulating in the blood in large proportion of human populations or in all human populations, as well as present receptors to the corresponding infectious agents. These antigens on such liposomes include, but are not limited to: α-gal epitope linked molecules binding the natural anti-Gal antibodies (Galili supra Immunology 2013), rhamnose linked to molecules binding natural anti-rhamnose antibodies (Chen et al. ASC Chem Biol 6:185, 2011), blood group A antigens binding anti-blood group A antibodies in subjects that have blood type B or O and blood group B antigens binding anti-blood group B antibodies in subjects that have A or O blood type. In addition, such antigens presented on liposomes and binding natural antibodies may include a variety of carbohydrate antigens against which natural antibodies were found in the blood of a large proportion of humans such as, but not limited to, those reviewed by Bovin N V (Biochemistry [Mosc] 78: 786, 2013) and tetanus toxoid (TT) which binds anti-tetanus toxoid antibody commonly present in humans. In a non-binding example, liposomes presenting any of these antigens as well as sialic acid epitopes and which are administered by inhalation to patients infected by influenza virus will land in the mucus and surfactant lining the epithelium of the respiratory tract, bind influenza virus via its sialic acid epitopes (SA epitopes) thus prevent infection of cells of the respiratory tract by the influenza virus. These liposomes further bind the natural antibody, or elicited antibody in the case of tetanus toxoid antigen, that interacts with the corresponding antigen the liposome presents, activate the complement system and thus recruit monocytes, macrophages and dendritic cells by the newly generated complement cleavage chemotactic peptides. The recruited monocytes, macrophages and dendritic cells will internalize these liposomes and the influenza virus bound to them as a result of interaction between the Fc receptors on these recruited cells and the Fc portion of the antibody bound to such liposomes. The immunogenic peptides of the internalized influenza virus are processed and presented by the recruited macrophages and dendritic cells functioning as APC which further transport these immunogenic peptides to the regional lymph nodes for eliciting a protective anti-influenza virus immune response.

In another non-binding example the liposomes present the sugar rhamnose linked to any spacer and interact with the natural anti-rhamnose antibody that is present in humans (Chen et al. 2011 supra) and also present SA epitopes. Following inhalation of these rhamnose/SA liposomes by symptomatic influenza patients the virus in the respiratory tract binds to the SA epitopes on these liposomes and the rhamnose epitopes bind anti-rhamnose antibodies. This rhamnose/anti-rhamnose interaction results in activation of complement, generation of chemotactic complement cleavage peptides such as, but not limited to C5a and C3a that induce rapid recruitment of monocytes, macrophages and dendritic cells. These recruited cells bind via their Fc receptors the Fc portion of the anti-rhamnose antibodies coating the liposomes and thus induce uptake and killing of the influenza virus bound to the SA epitopes on these liposomes. The internalized virus is further processed within the macrophage and dendritic cells, its immunogenic peptides are transported by the macrophages and dendritic cells functioning as APC to the regional lymph nodes and presented on these APC in association with MHC molecules for the activation of T cells specific to influenza virus.

In another embodiment, the liposomes used in this proposed therapy may present various antigens or epitopes which bind antibodies commonly found in humans and also present receptors that serve as binding sites or “docking receptors” for various viruses or bacteria. As a non-limiting example, sulfated glycosaminoglycans (GAGs) may serve as such receptors to various viruses which bind the virus similar to the binding of influenza virus to receptors comprised of glycans containing sialic acid (Olofsson and Bergström Ann Med. 37: 154, 2005).

### IV. The Natural Anti-Gal Antibody, α-Gal Epitopes and α-Gal Liposomes

The activity of the novel α-gal/SA liposomes is best explained by first describing the effects of the natural anti-Gal antibody interacting with α-gal liposomes, i.e., liposomes expressing multiple α-gal epitopes. Anti-Gal is the most abundant natural antibody in all humans constituting ˜1% of circulating immunoglobulins (Galili et al. J Exp Med, 1984 supra). Anti-Gal binds specifically to a carbohydrate antigen called the α-gal epitope with the structure Galα1-3Galβ1-4GlcNAc-R (Galili et al. J Exp Med 1985, supra). This antibody is produced throughout life in response to continuous antigenic stimulation by bacteria of the normal gastrointestinal flora (Galili et al. Infect Immun 56: 1730, 1988). Anti-Gal is naturally produced also in Old World monkeys (monkeys of Asia and Africa) and in apes, however, it is absent in other mammals (Galili et al. Proc. Natl Acad Sci USA 84: 1369, 1987). In contrast, other mammalian species, including nonprimate mammals (e.g. mice, rats, rabbits, dogs, pigs, etc.), as well as prosimians such as lemurs and New World monkeys (monkeys of South America), lack the anti-Gal antibody but they all produce its ligand, the α-gal epitope, by using a glycosylation enzyme called α1,3galactosyltransferae (α1,3GT) (Galili et al. Proc. Natl Acad Sci USA 1987, supra; Galili et al. J Biol Chem 263: 17755, 1988).

Since the natural anti-Gal antibody is present in large amounts in all humans who are not severely immunocompromised, it may be exploited for various clinical benefits. As described in U.S. Pat. No. 7,820,628 (Uri Galili—Inventor, indicated at the end of the references list), anti-Gal can be exploited by the use of micelles comprised only of pure α-gal glycolipid (i.e. lacking phospholipids) that are injected into solid tumors for conversion of the treated tumors into autologous anti-tumor vaccine (Galili et al. J Immunol 2007, supra). In addition, α-gal liposomes and the submicroscopic α-gal liposomes (also called α-gal nanoparticles) have been shown to induce accelerated healing of external and internal injuries, as described in the U.S. Pat. Nos. 8,084,057, 8,440,198 and 8,865,178 (Uri Galili—Inventor, indicated at the end of the references list) and which are described in the following publications: Galili et al. Burns supra, 2010; Wigglesworth et al. supra, J Immunol 2011; Hurwitz et al. Plastic Reconstruct Surgery 129: 242, 2012; Galili, The Open Tissue Engin Regen Med J 6: 1, 2013. This section describes the preparation and activities of α-gal liposomes and α-gal nanoparticles (i.e. α-gal liposomes and α-gal nanoparticles lacking SA-glycolipids) when applied in vivo. Sections below teach the preparation of α-gal/SA liposomes which are α-gal liposomes also comprised of SA-glycolipids. These sections further describe the activity of α-gal/SA liposomes in preventing infection of cells by influenza virus, in destruction of this virus by macrophages internalizing the virus when it is bound to α-gal/SA liposomes and in the in situ conversion of the internalized influenza virus into an effective influenza vaccine.

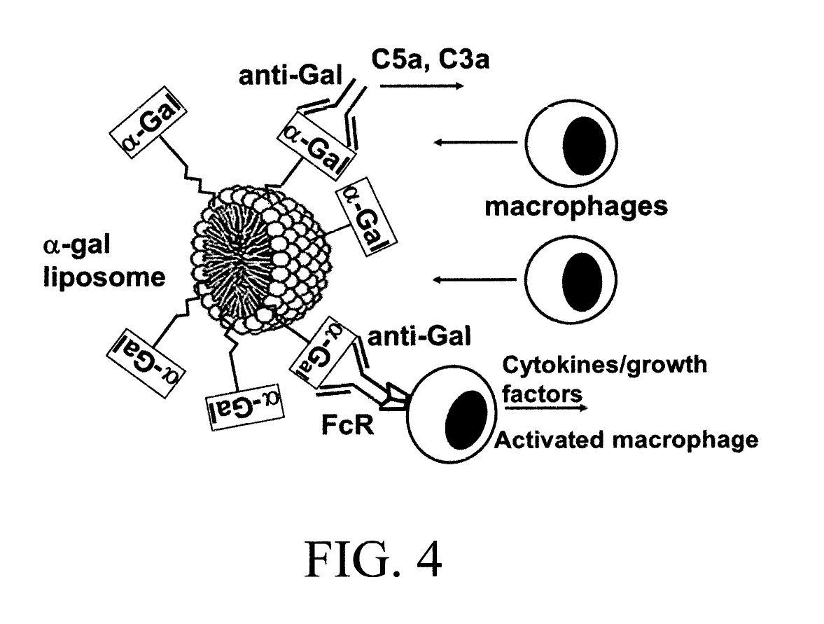

Previous studies by Galili and colleagues (Galili et al. Burns supra 2010; Wigglesworth et al. J Immunol supra, 2011; Hurwitz et al. Plastic Reconstruct Surgery supra 2012; Galili. The Open Tissue Engin Regen Med J supra 2013) indicated that the activity of the natural anti-Gal antibody can be harnessed in humans for clinical benefits by the use of α-gal liposomes. These liposomes have a structure similar to the α-gal/SA liposomes presented in FIGS. 2 and 3 with the exception that they lack SA-glycolipids. α-Gal liposomes can be prepared from various materials and they are characterized by presenting multiple α-gal epitopes. In a non-limiting example, α-gal liposomes are submicroscopic liposomes composed of glycolipids with multiple α-gal epitopes (α-gal glycolipids), phospholipids and cholesterol (Wigglesworth et al. J Immunol supra 2011). Since α-gal glycolipids comprise most of the glycolipids in rabbit red blood cell (RBC) membranes and since these RBC membranes are the richest known source of natural α-gal glycolipids in mammals (Galili et al. Proc Natl Acad Sci USA supra 1987; Egge et al. J Biol Chem 260: 4927, 1985, Galili et al. J Immunol supra 2007), rabbit RBC are a convenient natural source for preparation of α-gal liposomes (Wigglesworth et al. J Immunol supra 2011). For this purpose, glycolipids, phospholipids and cholesterol are extracted from rabbit RBC membranes in a mixture of chloroform and methanol (Galili et al. J Immunol supra 2007). The dried extract is sonicated in saline in a sonication bath to generate liposomes (size of approximately 1-50 μm) comprised of α-gal glycolipids, phospholipids and cholesterol and which present multiple α-gal epitopes of the glycolipids in the extract. These liposomes (referred to as α-gal liposomes) are further sonicated using a sonication probe into submicroscopic liposomes also called α-gal liposomes, which have the same composition as the α-gal liposomes, however their size range is 1-500 nm and preferably 10-300 nm. The α-gal liposomes suspension is further sterilized by filtration through a 0.2 μm filter. These submicroscopic α-gal liposomes are also referred to as α-gal nanoparticles (Galili, The Open Tissue Engin Regen Med J supra 2013). A schematic presentation of an α-gal liposome is illustrated in FIG. 4. This liposome has a wall of phospholipids such as but not limited to phosphatidyl choline in which α-gal glycolipids are anchored via the fatty acid tails of their ceramide portion. The glycolipid illustrated in FIG. 4 is capped with α-gal epitopes (α-gal in rectangles). α-Gal glycolipids in rabbit RBC membranes are of various lengths ranging from 5 to 40 carbohydrate units carrying 1-8 branches all capped with an α-gal epitope (Galili et al. 2007 supra 2007; Egge et al. J Biol Chem supra 1985; Hanfland et al. Carbohydrate Res 178: 1, 1988; Honma et al. J Biochem (Tokyo) 90:1187, 1981).

Overall, the number of α-gal epitopes on α-gal liposomes is very high, corresponding to ˜10¹⁵ α-gal epitopes per mg α-gal liposomes (Wigglesworth et al. J Immunol. Supra 2011). From 1 liter of rabbit RBC it is possible to prepare 3-4 grams of α-gal liposomes. The α-liposomes are highly stable since they contain no tertiary structures. Accordingly, no changes in expression of α-gal epitopes were found in α-gal liposomes kept at 4° C. or frozen for 4 years in comparison with freshly produced α-gal liposomes.

The α-gal liposomes can be made also in a synthetic form by the use of synthetic glycolipids such as, but not limited to synthetic α-gal epitopes linked to a lipid via a carbohydrate chain or via a linker, or both. Such synthetic glycolipids can be prepared by methods known to those skilled in the art. A phospholipid such as, but not limited to, phosphatidyl choline or other lipid suitable for liposomes formation, is dissolved in an organic solvent such as, but not limited to, methanol. A synthetic α-gal glycolipid is dissolved together with the phosphatidyl choline in methanol at a molar ratio such as, but not limited to 1:10 α-gal glycolipid:phospholipid. The mixture is dried in a rotary evaporator, or in any other drying device known to those skilled in the art. Subsequently, the dried mixture is sonicated to form synthetic α-gal liposomes comprised of phosphatidyl choline and α-gal glycolipid molecules. Synthetic α-gal liposomes may be prepared from any type of phospholipid and from synthetic glycolipids comprised of any kind of a lipid with one or more carbohydrate chains all or part of which carry α-gal epitopes. The α-gal epitopes may be linked to the lipid by a carbohydrate chain or by any spacer known to those skilled in the art. This linking of the α-gal epitope to the lipid portion is performed by methods known to those skilled in the art.

α-Gal liposomes were studies for their effects on wound healing and tissue regeneration following binding of the anti-Gal antibody. The studies on anti-Gal mediated acceleration of injury regeneration by α-gal liposomes cannot be performed in standard experimental animal models since, similar to all other nonprimate mammals, mice, rats, guinea-pigs, rabbits and pigs, all produce α-gal epitopes on their cells by the glycosylation enzyme α1,3galactosyltransferase (α1,3GT) and thus cannot produce the anti-Gal antibody, i.e. they are immunotolerant to the α-gal epitope (Galili et al. Proc Natl Acad Sci USA supra 1987; Galili et al. J Biol Chem, 1988, supra). In addition to Old World monkeys, the only two nonprimate experimental animal models which are suitable for anti-Gal studies are a 1,3 GT knockout mice (GT-KO mice) produced in the mid-1990s (Thall et al. J Biol Chem 270: 21437, 1995; Tearle et al. Transplantation 61: 13, 1996) and α1,3GT knockout pigs (GT-KO pigs) produced in the last decade (Lai et al. Science 295: 1089, 2002; Phelps et al. Science 299: 41, 2003). These two knockout animal models lack α-gal epitopes and can produce anti-Gal. Old World monkeys, which naturally produce the anti-Gal antibody can serve as animal models, as well.

V. Interaction of Anti-Gal Antibody with α-Gal Liposomes Induces Rapid Recruitment of Macrophages

Interaction between serum anti-Gal and α-gal epitopes on cells results in activation of the complement system. Transplantation of pig xenografts in monkeys is a demonstration of this complement activation. Binding of circulating anti-Gal antibody to the multiple α-gal epitopes on pig endothelial cells lining the blood vessels of pig kidney or heart xenografts, results in activation of the complement system that causes lysis of the endothelial cells, collapse of the vascular bed and hyperacute rejection of the xenograft within 30 minutes to several hours (Simon et al. Transplantation 56: 346, 1998; Xu et al. Transplantation 65: 172, 1998). A similar activation of complement occurs when serum anti-Gal binds to the multiple α-gal epitopes on α-gal liposomes. This complement activation results in the generation of chemotactic complement cleavage peptides that are among the most potent physiologic chemotactic factors. These include C5a, C4a and C3a complement cleavage peptides which induce rapid migration of macrophages into the site of α-gal liposomes application (Wigglesworth et al. J Immunol supra, 2011). In contrast to anti-Gal/α-gal epitopes interaction in xenotransplantation, no cells are damaged by anti-Gal/α-gal liposomes interaction since complement activation occurs on the surface of the liposomes presenting α-gal epitopes rather than on the surface of cells presenting α-gal epitopes.

In studies with α-gal liposomes injected intradermally into anti-Gal producing GT-K0 mice, mostly macrophages were found to be recruited following anti-Gal/α-gal liposomes interaction as a result of the generation of complement cleavage chemotactic peptides by this antibody/antigen interaction. Granulocytes were found at the injection site after 12 h and disappeared after 24 h, whereas macrophages reached the injection site within 24 h and continued migrating into that site for several days (Wigglesworth et al. J Immunol supra 2011). The identity of the migrating cells primarily as macrophages could be determined by immunostaining with the macrophage specific antibody (Wigglesworth et al. J Immunol supra 2011). The macrophages were found at the injection site for 14-17 days and completely disappeared within 21 days without changing skin architecture. No granulomas and no detrimental inflammatory responses were found in such α-gal liposomes injection sites. Similar recruitment of macrophages was observed with α-gal liposomes introduced subcutaneously in GT-KO mice within biologically inert polyvinyl alcohol (PVA) sponge discs containing the α-gal liposomes (Galili et al. Burns supra 2010). It is contemplated that binding of the anti-Gal antibody to α-gal epitopes on α-gal/SA liposomes described in FIG. 2 results in a similar effects on macrophages as those with the α-gal liposomes previously described (Galili et al. Burns supra 2010; Wigglesworth et al. J Immunol supra 2011) since the α-gal epitopes on α-gal/SA liposomes are identical to those on α-gal liposomes. Therefore, both types of liposomes interact with the anti-Gal antibody. It is further contemplated that binding of the anti-Gal antibody to α-gal/SA liposomes results in rapid recruitment of macrophages similar to that observed with α-gal liposomes because of a similar activation of complement as that resulting in recruitment of macrophages by α-gal liposomes that was previously described (Galili et al. Burns supra 2010; Wigglesworth et al. J Immunol supra 2011; Galili, The Open Tissue Engin Regen Med J supra 2013). Dendritic cells are also recruited to the α-gal liposomes as a result of the activity of complement cleavage chemotactic factors. This was shown in tumors injected with purified α-gal glycolipids in the form of micelles in GT-KO mice in which the anti-Gal antibody binds to α-gal epitopes on these glycolipids and induces recruitment of both macrophages and dendritic cells (Galili et al. J Immunol supra 2007).

### VI. Activation of Macrophages by Anti-Gal Coated α-Gal Liposomes

As indicated above, in situ binding of the natural anti-Gal antibody to α-gal epitopes on the α-gal liposomes results in activation of the complement system and thus, the generation of the complement peptide chemotactic factors as C5a, C4a and C3a which induce rapid recruitment of macrophages (Wigglesworth el al. J Immunol supra 2011). After the recruited macrophages reach the α-gal liposomes, the Fc “tails” of anti-Gal coating α-gal liposomes bind to Fc receptors (FcR) on these macrophages (Abdel-motal et al. VACCINE 27: 3072, 2009; Wigglesworth et al. J Immunol supra 2011). This extensive binding to FcR on macrophages was demonstrated by scanning electron microscopy with submicroscopic α-gal liposomes (also called α-gal nanoparticles) coated by anti-Gal and incubated in vitro with cultured macrophages of α1,3GT knockout pig origin (GT-KO pig). Multiple α-gal liposomes attach to the macrophages via the Fc/FcR interaction (Galili, The Open Tissue Engin Regen Med J supra 2013; Galili Tissue Engineering, Part B: Reviews, 21: 231, 2015; Galili J. Immunol. Res. Vol. 2015, Article ID 589648, 2015). In the absence of anti-Gal, no significant binding of α-gal liposomes to macrophages was observed. This Fc/FcR interaction induces the uptake of the α-gal liposomes with the immunocomplexed anti-Gal antibody into the macrophages (Abdel-motal et al. VACCINE 27: 3072, 2009). It is contemplated that α-gal/SA liposomes with bound influenza virus are internalized as a result of Fc/FcR interaction between anti-Gal bound to α-gal epitopes on these liposomes and macrophages as well as dendritic cells recruited by this anti-Gal/α-gal epitopes interaction.

α-Gal/SA Liposomes and their Preparation

The present invention teaches how to prepare α-gal/SA liposomes that have both the characteristics of α-gal liposomes interaction with the anti-Gal antibody and the ability to bind influenza virus via the interaction between hemagglutinin (HA) of the virus and sialic acid epitopes (SA epitopes) on α-gal/SA liposomes (FIG. 2). The α-gal/SA liposomes differ in structure from α-gal liposomes in that α-gal/SA liposomes present both α-gal epitopes and SA-epitopes, whereas α-gal liposomes present only α-gal epitopes. Therefore, α-gal/SA liposomes are a novel type of liposomes that differ from α-gal liposomes described in U.S. Pat. Nos. 8,084,057, 8,440,198 and 8,865,178 in that they also comprise SA glycolipids presenting SA epitopes and in that α-gal/SA liposomes are used for different purpose than the α-gal liposomes describes in these three US patents. Whereas α-gal liposomes are applied to external or internal injuries for accelerating healing of treated injuries, α-gal/SA liposomes are introduced by inhalation to the respiratory tract in order to bind infective influenza virus, thus inhibiting the ability of the virus from infecting respiratory epithelium cells. By targeting the virus bound to α-gal/SA liposomes for uptake by macrophages and dendritic cells functioning as APC the α-gal/SA liposomes further convert the infecting virus into effective endogenous vaccine that elicits a rapid protective immune response. The preparation α-gal/SA liposomes is similar to that of α-gal liposomes, however, instead of the liposomes having glycolipids with only α-gal epitopes as in α-gal liposomes, the α-gal/SA liposomes have glycolipids that carry α-gal epitopes and glycolipids that carry SA epitopes (i.e., glycolipids with sialic acid at the non-reducing end) (FIGS. 2 and 3).

α-Gal/SA liposomes may be prepared from natural material or from synthetic materials. In one embodiment, natural α-gal/SA liposomes may be prepared from phospholipids and α-gal glycolipids as well as SA-glycolipids and/or other glycans extracted from cells of eukaryotes of prokaryotes, including but not limited to membranes of mammalian red blood cells, using methods known to those skilled in the art. Non-limiting examples for membranes of mammalian red cells which may be the source of α-gal glycolipids, SA-glycolipids and phospholipids are rabbit red cells, bovine red cells and porcine red cells (Galili et al. Proc Natl Acad Sci USA supra 1987). One non-limiting example for as source of SA-glycolipids of phospholipids and SA-glycolipids for production of natural α-gal/SA liposomes may be human red cells. Human red cell SA-glycolipids may be mixed with α-gal glycolipids from other sources and with phospholipids for production of α-gal/SA liposomes. The mixture of α-gal glycolipids, SA-glycolipids and phospholipids is dried and sonicated in saline to generate liposomes of a size range but not limited to 0.001-100 μm, comprised of α-gal glycolipids, SA-glycolipids and phospholipids. The preparation of the natural α-gal/SA liposomes may also be performed by other methods known to those skilled in the art. The extracts used for the α-gal/SA liposomes may also include other molecules including but not limited to cholesterol and various glycans.

In another embodiment, synthetic α-gal/SA liposomes may be prepared by mixing in an organic solvent such as, but not limited to methanol, synthetic α-gal glycolipids, synthetic glycolipids with sialic acid at the non-reducing end (SA-glycolipids) and phospholipids (FIG. 3). The ratio of glycolipids to phospholipids (glycolipids:phospholipids) may be at the range of 1:100,000 to 100,000:1 but may not be limited to these ratios. The ratio of α-gal glycolipids to SA-glycolipids (α-gal glycolipids:SA-glycolipids) may be at the range of 1:100,000 to 100,000:1 but may not be limited to these ratios. In a preferred embodiment, the final ration for production of synthetic α-gal/SA liposomes may be 1:1:10 of α-gal glycolipids:SA-glycolipids:phospholipids, respectively. The extracts used for the α-gal/SA liposomes preparation also may include other molecules such as, but not limited to cholesterol and various glycans. The mixture of α-gal glycolipids, SA-glycolipids and phospholipids is dried in a rotary evaporator. The dried mixture is sonicated in saline to generate liposomes of a size range but not limited to 0.001-100 μm, comprised of α-gal glycolipids, SA-glycolipids and phospholipids. These liposomes present multiple α-gal epitopes and SA epitopes of the glycolipids (FIG. 3). These liposomes (referred to as α-gal/SA liposomes) are further sonicated by a sonication probe into submicroscopic liposomes that are also called α-gal/SA liposomes and, which have the same composition as the α-gal/SA liposomes, however their non-limiting size range is 1-500 nm and preferably 10-200 nm. The α-gal/SA liposomes suspension is further sterilized by filtration through a 0.2 μm filter which removes bacteria or protozoa from the α-gal/SA liposomes suspension, whereas the liposomes of the size of 200 nm can “squeeze’ through 0.2 μm pores.

α-Gal glycolipids to be used for production of synthetic α-gal/SA liposomes may be selected from the group consisting of but not limited to Galα1-3Gal-R, Galα1-2Gal-R, Galα1-6Gal-R and Galα1-6Glc-R. The α-gal epitopes may preferably be comprised of terminal galactosyl linked α1-3 to a penultimate N-acetyllactosamine, as Galα1-3Galβ1-4GlcNAc-R, or Galα1-3Galβ1-3GlcNAc-R where R is any carbohydrate chain or any linker linked to a ceramide, protein, proteoglycan or polymer. The α-gal epitopes on the α-gal/SA liposomes further may include oligosaccharides available from Dextra, but are not limited to: i) Galα-3Gal glycolipids: al-3 galactobiose (cat. # G203); linear B-2 trisaccharide (cat. # GN334); and Galili pentasaccharide (cat. # L537). Various other glycoconjugates with α-gal epitopes available from Dextra include for instance: Galα1-3Galβ1-4Glc-BSA (BSA—bovine serum albumin, cat. # NGP0330); Galα1-3Galβ1-4(3)-deoxyGlcNAc-HSA cat. # (HSA—human serum albumin, NGP2335); Galα1-3Galβ1-4GlcNAcβ1-HDPE (cat. # NGL0334); and Galα1-3Gal-BSA (cat. # NGP0203) all which may be linked to a lipid or to other materials that form α-gal/SA liposomes. Another non-limiting example is the Elicityl Galα1-3Gal Galili series of carbohydrate chains of various sizes carrying α-gal epitopes and having or lacking a linker, all of which may be linked to a lipid or to other materials that form liposomes. An additional non-limiting example is from Sigma-Aldrich “FSL-Galili-tri” (cat. # F9432) also produced by KODE Biothech (Auckland, NZ). The synthetic α-gal/SA liposomes may further present any epitopes that binds the anti-Gal antibody. Another non-limiting example is Carbohydrate Synthesis LTD manufacturing synthetic α-gal disaccharides cat. # BX501 (Galα1-3Gal-O-Me) and BX502 (Galα1-2Gal-O-Me) and trisaccharide cat. # C503 (Galα1-3Galβ1-4GlcNAc).

The sialic acid (SA) glycoconjugates on the α-gal/SA liposomes may include oligosaccharides with terminal SA at the non-reducing end and linked to ceramide or to proteins that may or may not be linked to a lipid tail. Such oligosaccharides with SA at the non-reducing end that may be linked to a lipid tail are available from Dextra, but are not limited to: i) 3′-Sialyl-N-acetyllactosamine (cat. #3′-SLN)-(cat. # SLN302), 3′-Sialyllactose (cat. #3′-SL)-(cat. # SL302), 6′-Sialyl-N-acetyllactosamine (6′-SLN)-(cat. # SLN306), 6′-Sialyllactose (6′-SL)-(cat. # SL306). Another non-limiting example is the Elicityl series of carbohydrate chains of various sizes carrying SA and having or lacking a linker and which may be linked to a lipid or to other materials that form liposomes such as but not limited to cat. # SAα2-3Galβ1-3GlcNAcβ1-3Galβ1-4Glc (cat. # GLY081), SAα2-3Galβ1-4GlcNAcβ1-3Galβ1-4Glc (cat. # GLY083), or SAα2-3Galβ1-3GlcNAcβ1-3Gal (cat. # GLY080). Synthetic SA oligosaccharides and synthetic SA glycolipids produced by other manufacturers are also suitable for production of α-gal/SA liposomes. In addition, natural or synthetic glycoproteins such as but not limited to human or other mammalian α2-acid glycoprotein, and fetuin, as well as natural or synthetic glycolipids which carry sialic acid at the non-reducing end of the carbohydrate chain are suitable for preparation of α-gal/SA liposomes and may be processed to be expressed by liposomes by methods known to those skilled in the art.

Based on studies on the affinity of influenza virus hemagglutinin (HA) to sialic acid epitopes on glycolipids (SA glycolipids), the terminal sialic acid may be linked to any penultimate carbohydrate and preferably to N-acetyllactosamine, as SA-Galβ1-(3)4GlcNAc-R, where R is any carbohydrate chain or any linker linked to a ceramide, protein, proteoglycan or polymer. The linkage between the terminal sialic acid and the penultimate carbohydrate may be any linkage, including but not limited to SAα2-6Galβ1-4GlcNAc-R and SAα2-3Galβ1-4GlcNAc-R or to a mixture of these two epitopes on each α-gal/SA liposomes (Rogers and Paulson Virology supra 1983; Suzuki et al. J Biol Chem supra 1986).

In another embodiment, α-gal/SA liposomes may be prepared from organic solvent extracts of mammalian red cell membranes that contain both α-gal glycolipids and SA glycolipids as well as phospholipids, such as, but not limited to bovine red cell membranes, porcine red cell membranes or rabbit red cell membranes (Chien et al. J. Biol. Chem. Supra 1978; Galili et al. Proc. Natl. Acad. Sci. USA supra, 1987), or from natural glycolipids that carry both α-gal epitope and SA epitope on the same glycolipid molecule (Watanabe et al. J Biol Chem supra, 1979) in addition to phospholipids. The phospholipids may originate from other natural or synthetic sources, as well.

### Mechanism for Anti-Influenza Virus Effects of α-Gal/SA Liposomes

Influenza viruses attach to susceptible cells via multivalent interactions of their hemagglutinin (HA) with SA epitopes comprised of sialyloligosaccharide moieties of cellular glycoconjugates (Wiley and Skehel Annu Rev Biochem 56: 365, 1987; Matrosovich and, Klenk Rev Med Virol 13: 85, 2003; Oshansky et al. PLoS One 6:e21183, 2011). Hemagglutinin is a trimeric glycoprotein that is present in multiple copies in the membrane envelope of influenza virus. In addition to the SA binding site, HA contains a fusion peptide and a transmembrane domain. The multivalent attachment to SA by multiple copies of trimetric HA triggers endocytosis of influenza virus that is subsequently contained in the endosome. Under the low interior pH of the endosome the HA undergoes conformational changes to insert the fusion peptide into the host membrane and further induce formation of a fusion pore that allows the release of the genome segments of influenza virus (Skehel and Wiley Annu Rev Biochem 69: 531, 2000). Because of the critical stage of HA binding to cell surface SA for enabling the virus entry step, inhibition of the HA/SA interaction was studied as potentially effective antiviral drugs of influenza viruses. Several studies demonstrated the ability of peptides carrying multiple synthetic SA epitopes, or of glycoproteins with such epitopes to inhibit infection of cells by influenza virus (Matrosovich and Klenk Rev Med Virol supra 2003; Rogers and Paulson Virology supra 1983; Suzuki et al. J Biol Chem supra 1986). However, this inhibition did not result in the destruction of the virus. Therefore, the therapeutic effect of such inhibitors for HA/SA interaction is limited. The present invention teaches how to combine the HA/SA inhibition step with a virus destruction step by macrophages as a result of administration of α-gal/SA liposomes by inhalation. Although knowledge of the mechanism(s) involved is not required in order to make and use the present invention, it is contemplated that the protective effects of the α-gal/SA liposomes against infective influenza virus are mediated by the following sequential processes (illustrated in FIG. 2):

1. Binding of Influenza Virus to Inhaled α-Gal/SA Liposomes—

A suspension of α-gal/SA liposomes in saline or any other physiologic buffer known to those skilled in the art is prepared in an inhaler, also called “nebulizer”, and preferably by a metered dose inhalers (MDI) at a possible concentration range of 1 μg/ml to 1.0 gm/ml and a preferable concentration range of 1.0 mg/ml to 100 mg/ml. The aerosolized α-gal/SA liposomes are inhaled by symptomatic patients upon detection or within few days after detection of influenza virus infection. The inhaled α-gal/SA liposomes “land” in the film of mucus covering the epithelium in the respiratory tract including, but not limited to the epithelium of the upper respiratory tract, the trachea, bronchi and bronchioles as well as in the film of surfactant within the alveoli. The influenza virus is also present in the symptomatic patient in the mucus and surfactant layers and it infects respiratory tract epithelium cells that have not been infected as yet. Influenza virus binds to the inhaled α-gal/SA liposomes as a result of the interaction between the multiple hemagglutinin (HA) trimers on the influenza viruses and SA epitopes on the α-gal/SA liposomes (FIG. 2). The binding of influenza virus to the α-gal/SA liposomes may be extensive enough to form aggregates between several α-gal/SA liposomes and multiple virions of influenza virus. Such aggregates (i.e., clumps) are formed by the same mechanism as that forming hemagglutination between red cells expressing SA and influenza virus. Thus the α-gal/SA liposomes act as a decoy binding the infecting influenza virus and inhibiting binding of influenza virus to the respiratory tract. Such decoy activity greatly decreases penetration of the infecting influenza virus into the respiratory epithelium cells.

2. Binding of Anti-Gal to α-Gal Epitopes on α-Gal/SA Liposomes Targets these Liposomes and the Influenza Virus Bound to them for Uptake by Macrophages and Dendritic Cells—

Anti-Gal antibodies of IgG, IgA and/or IgM classes that diffuse into the mucus lining the epithelium in the respiratory tract and into the surfactant in the alveoli bind to the α-gal epitopes on α-gal/SA liposomes. This antibody/antigen interaction activates the complement system in the mucus and surfactant of the respiratory tract, similar to most other antigen/antibody interactions. Among the products of this activation are chemotactic complement cleavage peptides such as, but not limited to C5a and C3a. These chemotactic factors induce rapid recruitment of macrophages and dendritic cells toward the α-gal/SA liposomes binding anti-Gal antibodies (FIG. 2). Once these recruited cells reach the α-gal/SA liposomes they bind these liposomes as a result of interaction between the Fc portion of anti-Gal antibodies coating the α-gal/SA liposomes (i.e., antibodies bound to the α-gal epitopes on α-gal/SA liposomes) and Fc receptors on the macrophages and dendritic cells. Such an interaction of α-gal/SA liposomes with macrophages is illustrated in FIG. 2 and was previously shown by scanning electron microscopy (Galili Tissue Engineering, Part B: Reviews, supra, 2015; Galili J. Immunol. Res. supra, 2015). Additional receptors that are contemplated to mediate binding of α-gal/SA liposomes to macrophages and dendritic cells are C3b receptors, also known as complement receptor type 1 (CR1) or CD35. These C3b receptors bind C3b complement deposits on the α-gal/SA liposomes as a result of complement activation by anti-Gal/α-gal epitopes interaction. The Fc/Fc receptor interactions and/or C3b/C3b receptor interactions activate the macrophages and dendritic cells to internalize the anti-Gal coated α-gal/SA liposomes in a manner similar to phagocytosis of any particulate material coated with its corresponding antibody. The influenza virus bound to the α-gal/SA liposomes is internalized by the macrophages and dendritic cells together with these liposomes. The virions of influenza virus internalized into macrophages and dendritic cells together with the α-gal/SA liposomes are further killed within the lysosomes of the macrophages and dendritic cells that fuse with the phagosomes in these cells. Killing of influenza virus internalized by macrophages and dendritic cells has been reported in several studies (Ionidis et al. J Virol 86: 5922, 2012; Reading et al. J Virol 74: 5190, 2000; Peschke et al. Immunobiology 189: 340, 1993). This mechanism of influenza virus killing by phagocytosis of virus complexed with the α-gal/SA liposomes is unique among methods used for decreasing virus infection of the respiratory tract epithelium. Other therapeutic methods affecting viral neuraminidase or preventing HA/SA interaction do not involve an active step of killing of the virus by its antibody mediated uptake into macrophages. In contrast, the treatment involving α-gal/SA liposomes inhalation specifically targets the virus for active uptake by macrophages that bind the α-gal/SA liposomes via Fc/Fc receptor interaction (FIG. 2). In the absence of α-gal/SA liposomes, the accidental endocytosis of influenza virus by relatively few macrophages in the mucus lining the epithelium of the respiratory tract results in ineffective destruction of the virus and progression of the disease into a prolonged infection which, in some cases may be life threatening.

3. Conversion of the Phagocytosed Influenza Virus into an Effective Vaccine—

The mounting of a physiologic protective immune response in humans against the infective influenza virus is relatively slow because of poor uptake, processing and presentation of the virus by relatively few antigen presenting cells (APC) such as dendritic cells and macrophages at early stages of the disease. Following inhalation of α-gal/SA liposomes, both macrophages and dendritic cells migrate toward the α-gal/SA liposomes as a result of complement activation and migration along chemotactic gradients of complement cleavage peptides. Such migration was previously observed in tumors injected with α-gal glycolipids that insert into tumor cell membranes and bind the anti-Gal antibody (Galili et al. J Immunol supra 2007). The Fc/Fc receptor interaction with anti-Gal coating α-gal/SA liposomes occurs both in macrophages and in dendritic cells. Therefore, uptake of the virus is effective in both macrophages and dendritic cells. As a result of this uptake the infecting virus can be internalized and processed by APC and transported by these APC to regional lymph nodes at early stages of the disease. Both macrophages and dendritic cells process the influenza virus proteins into peptides that are presented on cell surface class I and class II MHC molecules. Within the lymph nodes, the macrophages and dendritic cells further present the processed and presented peptides to T helper cells (CD4+ T cells) and to cytotoxic T cells (CD8+ T cells). The influenza virus specific CD4+ helper T cells are activated by influenza virus peptides presented on class II MHC molecules and help influenza virus specific B cell clones to expand and differentiate into plasma cells that produce protective antibodies such as, but not limited to anti-HA antibodies which neutralize the infecting virus. The influenza virus specific CD8+ T cells are activated by influenza virus peptides presented on class 1 MHC molecules. These T cell clones expand and mature into cytotoxic T cells (CTL) which are capable of killing cells that are infected by influenza virus. Such CTL mediated killing of virus infected cells prevents further propagation of the virus and prevention of increase in influenza virus burden within the infected patient. Thus, the inhalation of α-gal/SA liposomes results in rapid uptake of the virus by recruited APC and acceleration of the induction of protective humoral and cellular immune responses that may thwart the progression of the influenza virus infection, decrease the disease period and avoid morbidity and mortality.

In the absence of α-gal/SA liposomes, the uptake of the influenza virus by macrophages and dendritic cells is much less extensive than in the presence of α-gal/SA liposomes for the following reasons: 1. The number of the APC (i.e., macrophages and dendritic cells) in the mucus lining the epithelium of the respiratory tract is much lower than the number of the APC following recruitment by complement cleavage chemotactic peptides that are generated as a result of anti-Gal binding to α-gal/SA liposomes, and 2. The uptake of the virus by each APC is much lower in the absence of α-gal/SA liposomes as it is mediated by random accidental endocytosis. In contrast, the active targeting of the influenza virus bound to the α-gal/SA liposomes, is mediated by interaction of Fc portion of anti-Gal on these liposomes and Fc receptors on dendritic cells and macrophages and/or by interaction of C3b deposits on the α-gal/SA liposomes and C3b receptor on dendritic cells and macrophages functioning as APC. As described in Example 4 of the Experimental section of this invention application, the efficacy of the anti-Gal/α-gal epitope interaction in targeting influenza virus to APC results in ˜100 fold increase in the immune response against influenza virus.

It is further contemplated that α-gal liposomes also expressing corresponding “docking” receptors (i.e., cell surface receptors enabling the virus to adhere to cells before penetrating them) of various respiratory viruses will decrease infectivity of such viruses by functioning as decoys and induce their anti-Gal mediated targeting of viruses bound to such liposomes to APC such as dendritic cells and macrophages. The mechanism for decreasing the infectivity of various respiratory viruses will be similar to that described in FIG. 2 for α-gal/SA liposomes decreasing infectivity of influenza virus, with the difference that the receptor binding the virus may not be SA epitope but other carbohydrate or non-carbohydrate epitopes which are specific for binding the virus causing the treated infection.

In addition, it is further contemplated that dry powdered inhalers (DPIs) may deliver a dry powder consisting of biodegradable particles, or nanoparticles that present on their surface both α-gal epitopes and SA epitopes. Following their inhalation, such particles, or nanoparticles that present on their surface both α-gal epitopes and SA epitopes will function similar to α-gal/SA liposomes by binding of influenza virus to the SA epitopes on the particles, or nanoparticles landing in the mucus and surfactant of the lungs and bind of anti-Gal antibody to the α-gal epitopes on the particles and nanoparticles. This anti-Gal/α-gal epitopes interaction activates the complement system which generates complement cleavage chemotactic peptides that induce chemotactic recruitment of macrophages and dendritic cells. Binding of the recruited macrophages and dendritic cells to these anti-Gal coated particles via the interaction between the Fc receptors on the macrophages and Fc portion of anti-Gal antibody immunocomplexed to said particles induces uptake of the particles and of the attached influenza virus by the macrophages and dendritic cells, processing and presentation of the virus immunogenic peptides by these macrophages. This uptake of the particles and influenza virus bound to them will inhibit binding of the virus to respiratory epithelium cells. Furthermore, the macrophages and dendritic cells internalizing and processing the virus, transport of the presented influenza virus immunogenic peptides to the regional lymph nodes, for eliciting a rapid and effective protective immune response against the infecting influenza virus, by processes similar to those described above and in FIG. 2 for inhalation of α-gal/SA liposomes.

### EXPERIMENTAL

The following examples are provided in order to demonstrate and further illustrate certain preferred embodiments and aspects of the present invention and are not to be construed as limiting the scope thereof. These examples describe the interaction of the anti-Gal antibody and of influenza virus with α-gal/SA liposomes. The examples further describe interaction with α-gal epitopes on α-gal liposomes as evaluated in the experimental animal model of α1,3galactosyltransferase knockout mice (referred to as GT-KO mice) which lack α-gal epitopes and produce the anti-Gal antibody. The quantification of in vivo recruitment was performed in GT-KO mice (Thall et al. J Biol Chem supra 1995) producing the anti-Gal antibody. In wild type mice, as in other nonprimate mammals the α1,3GT gene (also called GGTA1 gene) encodes for the α1,3galactosyltransferase (α1,3GT) enzyme that synthesizes α-gal epitopes on glycolipids, glycoproteins and proteoglycans (Galili et al. J Biol Chem supra 1988). In GT-KO mice the α1,3GT gene was disrupted by gene “knockout” technology and thus these mice do not produce α-gal epitopes and are not immunotolerant to them (LaTemple and Galili Xenotransplantation 5: 191, 1998). The mice were induced to produce the anti-Gal antibody at titers similar to those in humans by pre-immunization with 50 mg pig kidney membranes since these membranes present multiple α-gal epitopes (Galili et al. J Immunol supra 2007).

In the experimental disclosure which follows, the following abbreviations apply: kDa (kilodalton); rec. (recombinant); N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); C (degrees Centigrade); ELISA (enzyme linked immunosorbent assay); mAb (monoclonal antibody); APC (antigen presenting cell); CTL (cytotoxic T lymphocyte); DC (dendritic cells); flu (influenza); HA (hemagglutinin); HAU (hemagglutination units); NA (neuraminidase); NP (nucleoprotein); influenza virus PR8 (A/Puerto Rico/8/34-H1N1 virus); Th (helper T); and IFNγ (interferon-γ).

### Example 1

### Interaction of the Natural Anti-Gal Antibody and of Influenza Virus with α-Gal/SA Liposomes

The α-gal/SA liposomes present two types of carbohydrate epitopes which are reactive in the process of inhibiting influenza virus infection of epithelial cells in the respiratory tract: 1. Sialic acid (SA) epitopes which bind the envelope hemagglutinin (HA) of the influenza virus, 2. α-Gal epitopes that bind the natural anti-Gal antibody, that activate the complement system for recruitment of macrophages and dendritic cells and targets the α-gal/SA liposomes and influenza virus bound to these liposomes for uptake by macrophages and dendritic cells via Fc/Fc receptor interaction and C3b/C3b receptor interaction. A schematic illustration of SA epitopes and of α-gal epitopes is included in FIG. 1. The binding of influenza virus to SA epitopes on red cells or on glycoconjugates has been demonstrated in multiple studies including: 1. Removal of SA from fowl or mammalian red cells by enzymatic treatment with neuraminidase prevents the subsequent binding of influenza virus to red cells devoid of SA (Wiley and Skehel Annu Rev Biochem supra 1987; Skehel and Wiley Annu Rev Biochem supra 2000). 2. Preincubation of influenza virus with glycoproteins or glycopeptides carrying carbohydrate chains with terminal SA blocks the HA of the virus from binding to SA on red cells and thus prevents hemagglutination of the red cells (Baum and Paulson Acta Histochem Suppl. 40:35, 1990; Mochalova et al. Virology 313: 473, 2003).

The present example (Example 1) demonstrates the interaction binding of influenza virus to SA epitopes on α-gal/SA liposomes and the binding of anti-Gal antibody to α-gal epitopes on α-gal/SA liposomes. These liposomes were produced as previously partly described (Wigglesworth et al. J Immunol supra 2011). Briefly, rabbit red cell membranes were subjected to overnight extraction by incubation with constant stirring in chloroform:methanol at a 1:2 ratio. This results in solubilization of glycolipids, phospholipids and cholesterol which are subsequently dried in a rotary evaporator. The proteins are denatured and removed by filtration. A large proportion of the extracted glycolipids is comprised of glycolipids with one or multiple α-gal epitops (α-gal glycolipids) (Galili et al. J Immunol supra 2007). Glycolipids with SA epitopes (SA glycolipids) were obtained by a similar extraction process from human red cell membranes. The extracts were dried individually or mixed at a ratio of 10:1 rabbit:human red cell membranes extracts. The dried extracts are sonicated in saline into liposomes. Since the liposomes prepared from mixture of rabbit and human red cell glycolipids carry α-gal epitopes and SA epitopes, these liposomes were designated α-gal/SA liposomes. Liposomes made of human red cell membranes extracts has SA epitopes, but lack α-gal epitopes were designated SA liposomes.

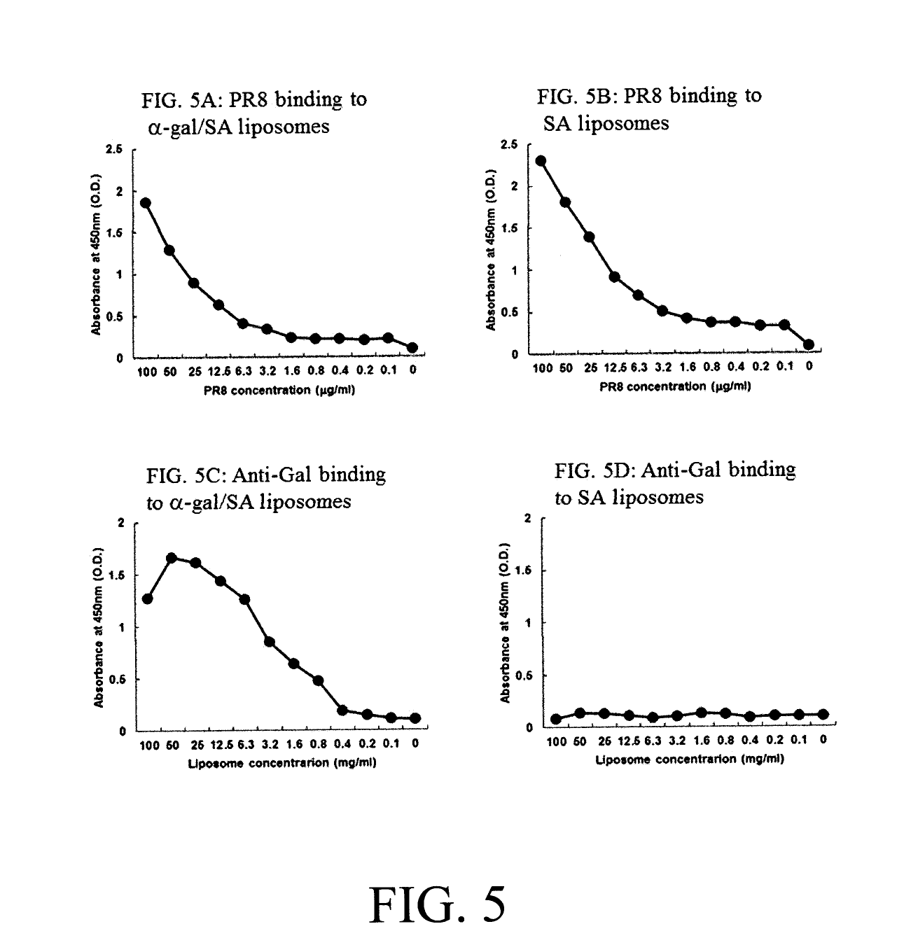

For evaluation of binding of influenza virus PR8 (A/Puerto Rico/8/34-H1N1) to SA epitopes on liposomes, the liposomes were plated in ELISA wells at 10 μg/ml in PBS (50 μl per well). The plates were dried overnight in a chemical hood to adhere the liposomes to the wells then blocked with 1% BSA in PBS. The PR8 virus was serially diluted at 1:2 starting at 100 μg/ml in the wells. After 2 hour incubation the wells were washed and mouse serum containing anti-PR8 antibodies (diluted 1:500) was added to each well for one hour, then the plates were washed and the binding of the virus to the liposomes coating the wells was determined by one hour incubation with anti-mouse IgG F(ab)₂ coupled with horse radish peroxidase (HRP) (Cappel, diluted 1:1000) as the secondary antibody. After additional washes, BD OptEIA TMB Substrate Reagent Set (BD 555214) was added for color reaction by the peroxidase linked to the secondary antibody. The light absorption was measured at 450 nm. As shown in FIGS. 5A and 5B, influenza PR8 virus bound both to α-gal/SA liposomes and to SA liposomes, respectively, indicating that both types of liposomes present SA epitopes capable of interacting with hemagglutinin (HA) of the virus and binding the PR8 virus.

For the evaluation of anti-Gal antibody binding to α-gal epitopes on the liposomes, various concentrations of liposomes were plated in ELISA wells as serial two fold dilutions starting at 100 μg/ml in PBS (50 μl per well). The plates were dried overnight then blocked with 1% bovine serum albumin (BSA) in PBS. Subsequently, the monoclonal anti-Gal IgM antibody, called M86 (Galili et al. Transplantation, 65:1129, 1998), was added to each well. The antibody binding determined by anti-mouse IgM-HRP (1:1000) as secondary antibody and TMB peroxidase substrate for color reaction. As shown in FIG. 5C, anti-Gal antibody readily bound to α-gal epitopes on α-gal/SA liposomes, but this antibody did not bind to SA liposomes made of human red cell membranes since human red cells completely lack α-gal epitopes (FIG. 5D) (Galili et al. Proc Natl Acad Sci USA supra 1987).

The observations in Example 1 indicate that α-gal/SA liposomes express both α-gal epitopes which bind the anti-Gal antibody and SA epitopes that bind influenza virus.

### Example 2

### Inhibiting Influenza Virus Progression of Infection by α-Gal/SA Liposomes Inhalation

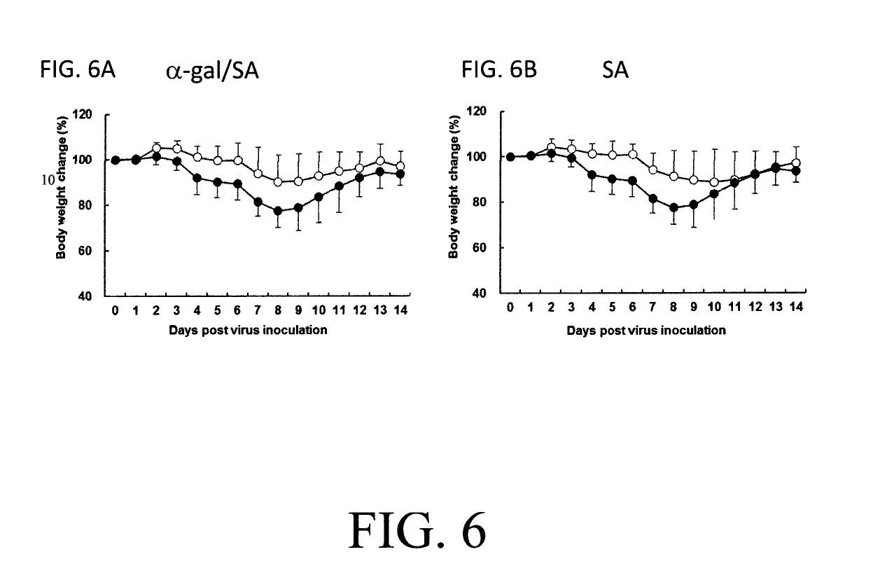

The objective of the experiment in Example 2 was to determine in a mouse experimental model whether inhalation of α-gal/SA liposomes can slow or inhibit the progression of influenza virus infection. For this purpose, anti-Gal producing GT-KO mice received intranasal inoculation of 50 μl of a sub-lethal dose of A/Puerto Rico/8/34-H1N1 influenza virus (PR8 virus). Subsequently, the mice are subjected to inhalation of α-gal/SA liposomes, SA liposomes or saline and monitored for 2 weeks for body weight and clinical signs. The inhalation was performed 3 times on Days 0-3, twice on Days 4 and 5 and once on Days 6 and 7. Decreasing body weight in the monitored mice indicated progression of the influenza virus infection in the lungs, whereas increase in body weight indicated recovery from the virus infection As shown in FIG. 6A mice that were infected with PR8 virus and inhaled saline displayed decrease in their body weight already by Day 3 due to the influenza virus infection (). By Day 8, the infected mice lost as much as 25% of the body weight and subsequently they slowly regain the body weight mice. However, even after 14 days their body weight does not fully return to the pre-infection weight. In contrast, mice treated in FIG. 6A by inhalation of α-gal/SA liposomes post PR8 infection (◯) did not display any loss of body weight before Day 7 and did not lose more than 10% of the body weight on Day 8. Subsequently, the mice fully regained 100% of their body weight by Day 13. The decreased infection of mice treated with the α-gal/SA liposomes vs. that in control mice is likely to be primarily the result of PR8 virus binding to the SA epitopes on the liposomes. This can be inferred from the study in FIG. 6B describing the weight loss in mice inhaling SA liposomes, instead of α-gal/SA liposomes. The extent of body weight loss in mice treated by inhalation with SA liposomes was similar to that described in FIG. 6A in mice treated by inhalation of α-gal/SA liposomes. Nevertheless, the findings that body loss in mice treated with SA liposomes peaked on Day 10 whereas that in α-gal/SA liposomes treated mice peaked on Day 8 and the somewhat faster regain of body weight in the latter mice, both suggest that the binding of anti-Gal to α-gal epitopes on the α-gal/SA liposomes contributed to the improved inhibition of the PR8 virus infection in comparison to the inhibition in SA liposomes treated mice.

Overall, the observation in Example 2 indicate that inhalation of α-gal/SA liposomes by mice infected intranasally with influenza virus results in significant decrease in the severity of the virus infection in comparison with the infection in control mice that are not treated by liposomes inhalation.

### Example 3

### Recruitment of Macrophages by α-Gal Liposomes in GT-KO Mice

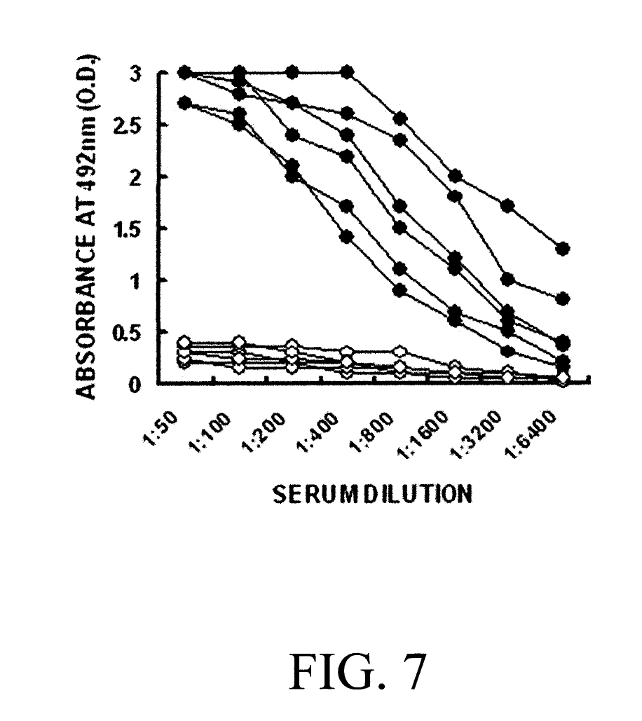

The purpose of this example is to determine whether the binding of the anti-Gal antibody to α-gal epitopes on α-gal/SA liposomes can induce in vivo recruitment of macrophages due to complement activation, as illustrated in FIG. 2. The quantification of in vivo recruitment of macrophages was performed in α1,3galactosyltransferase knockout (GT-KO) mice (Thall et al. J Biol Chem supra 1995) producing the anti-Gal antibody. The study was performed with liposomes prepared from rabbit red cell membranes that express multiple α-gal epitopes. Since α-gal glycolipids comprise most of the glycolipids in rabbit red cell membranes and since these red cell membranes are among the richest known sources of natural α-gal glycolipids in mammals (Galili et al. Proc Natl Acad Sci USA supra 1987; Egge et al. J Biol Chem 260: 4927, 1985, Galili et al. J Immunol supra 2007), rabbit red cells are a convenient natural source for preparation of liposomes presenting multiple α-gal epitopes (1×10¹⁵ α-gal epitopes/mg liposomes). These liposomes have been referred to as α-gal liposomes (Galili et al. BURNS supra, 2010; Wigglesworth et al. J Immunol supra 2011). Indeed the anti-Gal antibody produced by GT-KO mice readily binds to the many α-gal epitopes on these α-gal liposomes. As shown in FIG. 7, anti-Gal containing sera of GT-KO mice were placed in serial two fold dilutions in the ELISA wells coated with the rabbit red cell α-gal liposomes as solid phase for 2 hours. The wells were washed and a secondary antibody goat anti-mouse IgG linked to horseradish peroxidase (HRP) was added for 1 h. The wells were washed and color reaction was developed for 5 min with ortho-phenylene diamine (OPD, 1 mg/ml, Sigma Co.). GT-KO mouse anti-Gal antibody in the sera bound to the rabbit red cell liposomes () in accord with the expression of multiple α-gal epitopes on these liposomes. In contrast, no antibody binding was observed when the sera were of wild-type (WT) mice which produce α-gal epitopes on their cells and thus, are incapable of producing the anti-Gal antibody (◯) (FIG. 7). These observations imply that the anti-Gal antibody produced in GT-KO mice binds effectively to the multiple α-gal epitopes on α-gal liposomes.

Recruitment of macrophages in vivo was studied with biologically inert polyvinyl alcohol (PVA) sponge discs of 10 mm in diameter and 2.5 mm thick that contained 10 mg α-gal liposomes in saline. The PVA sponges were implanted subcutaneously in the dorsal region of GT-KO mice. The PVA sponge discs were retrieved at various days and squeezed repeatedly in PBS to obtain and characterized the infiltrating cells. The infiltrating cells on days 3-9 had a morphology of macrophages.

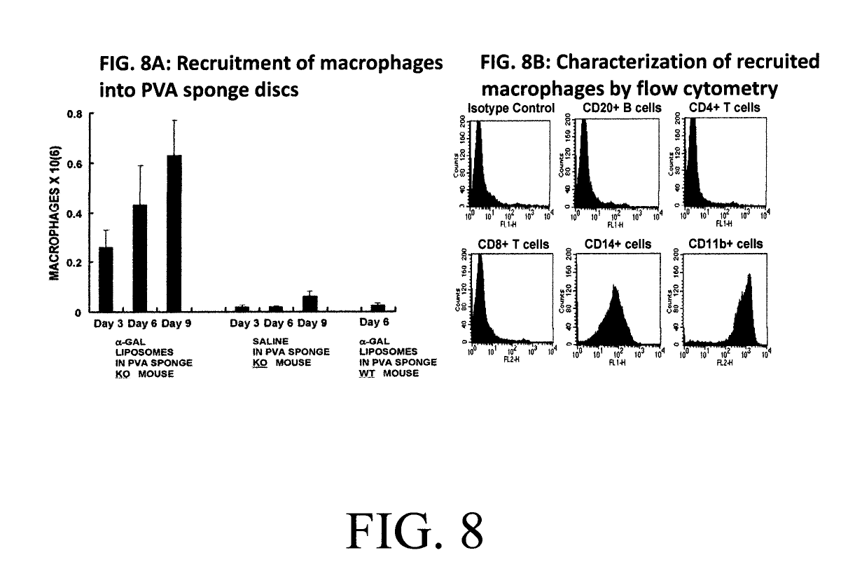

Quantification of the infiltrating macrophages in PVA sponges indicated that the number of recruited cells was directly related to the length of the implantation period. PVA sponges obtained on Day 3 contain 0.2×10⁶ macrophages in a volume of 0.1 ml whereas those obtained on Days 6 or 9, each contained 0.4×10⁶ and 0.6×10⁶ cells, respectively (FIG. 8A). In sponges containing only saline and no liposomes, the number of infiltrating macrophages was <10% of that found in α-gal liposomes containing sponges (FIG. 8A). A similar analysis was performed with PVA sponges containing α-gal liposomes that were implanted in wild type (WT) mice, i.e. in mice lacking the anti-Gal antibody. Very small numbers of cells migrating into sponge on Day 6 post implantation were observed in WT mice (FIG. 8A). This observations further imply that in the absence of anti-Gal no recruitment of macrophages occurs because there is no complement activation and no generation of complement cleavage chemotactic peptides.

Definite characterization of the recruited cells as macrophages was achieved by flow cytometry analysis. Infiltrating cells were retrieved from explanted PVA sponges at several time points, counted and immunostained for various cell surface markers. FIG. 8B demonstrates the flow cytometry analysis of immunostained cells retrieved on Day 6. Almost all infiltrating cells were macrophages, as indicated by expression of CD11b and CD14 macrophage markers. No significant numbers of B cells (CD20⁺) or T cells (CD4⁺ and CD8⁺) were detected. The same immunostaining patterns were observed with cells retrieved on Days 3 and 9 (not shown).

Overall, these findings in Example 3 demonstrate the very effective mechanism of macrophage recruitment as a result of the antibody-antigen interaction between the anti-Gal antibody and α-gal liposomes. These findings further imply that trapping of inhaled α-gal liposomes within the mucus of the respiratory tract will result in binding of anti-Gal to these liposomes and rapid recruitment of macrophages. Since α-gal/SA liposomes present multiple α-gal epitopes which are the same as the α-gal epitopes on α-gal liposomes (FIGS. 2 and 3), it is contemplated that binding of the natural anti-Gal antibody to inhaled α-gal/SA liposomes also activates the complement system and induces rapid recruitment of macrophages toward the α-gal/SA liposomes. This recruitment occurs concomitantly with the binding of influenza virus via its hemagglutinin (HA) to SA epitopes on these α-gal/SA liposomes.

### Example 4

### Increased Immunogenicity of Influenza Virus Presenting α-Gal Epitopes and Targeted to APC by the Anti-Gal Antibody

Example 4 describes the ability of the anti-Gal antibody to increase the immunogenicity of influenza virus processed to express α-gal epitopes. This example supports the proposed mechanism described in FIG. 2, claiming that influenza virus bound to α-gal/SA liposomes will function as a more potent vaccinating virus eliciting an effective protective anti-virus immune response than the virus infecting the respiratory tract and eliciting a physiologic immune response in patients that are not treated with α-gal/SA liposomes. This increased immunogenicity in Example 4 was achieved by anti-Gal mediated targeting of α-gal epitopes expressing virus to antigen presenting cells (APC) such as macrophages and dendritic cells. Although knowledge of the mechanism(s) involved is not required in order to make and use the present invention, it is contemplated that a similar anti-Gal mediated increase in immunogenicity occurs with influenza virus that is bound to SA epitopes on α-gal/SA liposomes and therefore is targeted by anti-Gal to APC. The key factor in increasing the immunogenicity of influenza virus, as well as in increased immunogenicity of other viruses is the targeting of the virus for extensive uptake (i.e. internalization) by APC such as macrophages and dendritic cells. The results presented in Example 4 are of experiments in which influenza virus is enzymatically processed to present α-gal epitopes, thus it binds anti-Gal and is targeted for increased uptake by APC.

The only difference between an immunization with α-gal epitopes expressing influenza virus, as that in Example 4 and immunization with influenza virus bound to α-gal/SA liposomes as in the present invention, is the site of α-gal epitopes presentation. In Example 4 the targeting to APC is mediated by anti-Gal bound to α-gal epitopes on influenza virus (Abdel-motal et al. J Virol, supra, 2007), whereas in the present invention the targeting is mediated by anti-Gal bound to α-gal epitopes on the α-gal/SA liposomes, to which the influenza virus is bound via SA epitopes on the liposomes (FIG. 2). In both methods, however, anti-Gal induces extensive uptake of influenza virus into APC, by binding to α-gal epitopes whether these epitopes are on the virus, or on the α-gal/SA liposomes. Once the virus is taken up by APC in each of these methods, the intracellular pathways within the APC are similar for the influenza virus antigens and include processing of immunogenic virus peptides and their presentation on Class I and Class II MHC molecules for the activation of influenza virus specific CD8+ and CD4+ T cells respectively within the regional (draining) lymph nodes. Thus, demonstration of increased immunogenicity in influenza virus expressing α-gal epitopes implies a similar increased immunogenicity of influenza virus that is bound to α-gal/SA liposomes inhaled by patients infected with the influenza virus.

Synthesis of α-Gal Epitopes on Influenza Virus PR8—

The study was performed on the experimental influenza virus strain PR8 which is infective in mice (Abdel-motal et al. J Virol 81: 9131, 2007). A process for achieving expression of α-gal epitopes on influenza virus by in vitro incubation with recombinant α1,3GT and with UDP-Gal has been described in U.S. Pat. Nos. 5,879,675 and 6,361,775 (U. Galili inventor). Synthesis of ˜3000 α-gal epitopes per virion on PR8 virus produced in embryonated eggs (i.e. lacking α-gal epitopes) was performed by incubation of the virus in a solution of 30 μg/ml recombinant (rec.) α1,3GT and 0.1 mM UDP-Gal (uridine diphosphate-galactose) as a sugar donor (Abdel-motal et al. J Virol supra 2007). The enzyme transfers the galactose from UDP-Gal and links it in a Galα1-3 linkage to the N-acetyllactosamines (Galβ1-4GlcNAc-R) of the multiple HA carbohydrate chains to generate α-gal epitopes. This reaction is identical to that which naturally occurs within the Golgi apparatus of nonprimate mammalian cells. Synthesis of the α-gal epitopes on HA of PR8 was confirmed by binding of monoclonal anti-Gal antibody to the HA of the processed virus in Western blots and ELISA (Abdel-motal et al. J Virol supra 2007). The PR8 virus presenting α-gal epitopes is called PR8_{αgal} virus.

Increased Influenza Virus Specific T Cell Activation in Mice Immunized with PR8_{αgal} Virus as Measured by ELISPOT

Increased activation of influenza virus specific T cells following vaccination with PR8_{αgal} virus, in comparison to vaccination with PR8 virus was studied in the experimental animal model of anti-Gal producing GT-KO mice. GT-KO mice producing anti-Gal were immunized twice in bi-weekly intervals with 1 μg inactivated PR8_{αgal} virus or with inactivated PR8 virus (i.e. virus lacking α-gal epitopes). The inactivation was achieved by incubation of the virus for 45 min at 64° C., and confirmed by demonstration of a complete loss of chicken red blood cell (ChRBC) hemagglutinating activity. The inactivated virus was injected subcutaneously with Ribi© (trehalose dicorynomycolate) adjuvant (Abdel-motal et al. J Virol supra 2007).

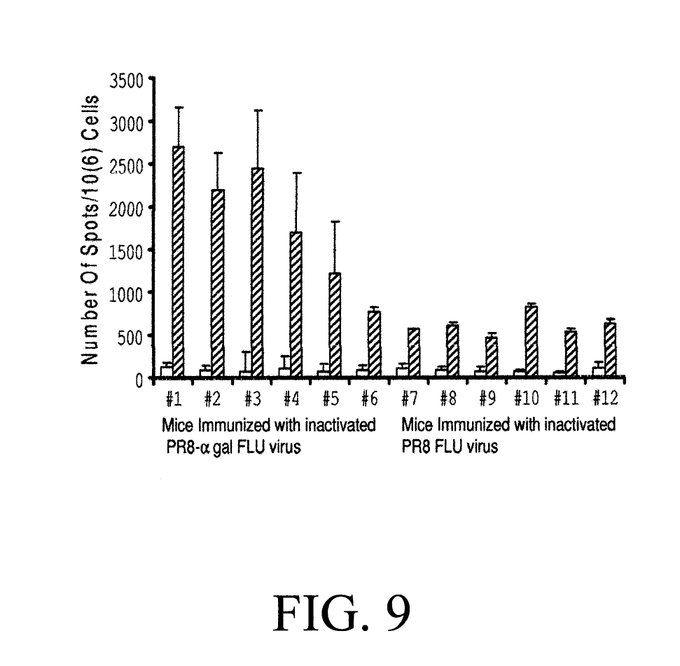

The mice were studied for anti-PR8 immune response, 4 weeks after the second immunization. PR8-specific T cells were detected in the spleens of the immunized mice by ELISPOT assays, which measured secretion of interferon-γ (IFNγ) following stimulation in vitro by PR8 antigens presented on dendritic cells. For this purpose, GT-KO mouse dendritic cells were incubated (i.e., pulsed) for 24 h with inactivated PR8 influenza virus, then co-incubated for an additional 24 h with spleen lymphocytes from the mice immunized with PR8_{αgal} or with PR8 virus. PR8 specific T cells, stimulated by dendritic cells presenting immunogenic PR8 peptides, secrete IFNγ which binds to the anti-IFNγ antibody coating the bottom of the ELISPOT well at the secretion site. The number of T cells that secrete IFNγ in the absence of stimulatory PR8 did not exceed 50 per 10⁶ lymphocytes in any of the mice tested (open columns in FIG. 9). In mice immunized twice with the inactivated unprocessed PR8 virus (mice #7-12), the number of activated virus specific T cells ranged between 400 and 700 per 10⁶ lymphocytes, with an average±standard deviation of 510±103 spots/10⁶ cells (hatched columns in FIG. 9). The number of PR8 specific T cells in 4 of the 6 mice immunized with PR8_{αgal} (mice #1-4) was several fold higher and ranged between 1650 and 2510 per 10⁶ lymphocytes. In the remaining two mice the number of these T cells was 750 and 1200 per 10⁶ lymphocytes. The average±standard deviation of the ELISPOT values in the mice immunized with PR8_{αgal} was 1800±760. These studies indicate that influenza virus is much more immunogenic than influenza virus lacking α-gal epitopes. Thus, if anti-Gal binds to α-gal epitopes on the vaccinating virus, it enhances viral opsonization (e.g., targeting the vaccinating virus for effective uptake by APC), resulting in a much more effective activation of T cells against influenza virus antigens.

Increased PR8 Specific CD8+ and CD4+ T Cell Responses Following PR8_{αgal} Immunization as Measured by Intracellular Cytokine Staining (ICS)—

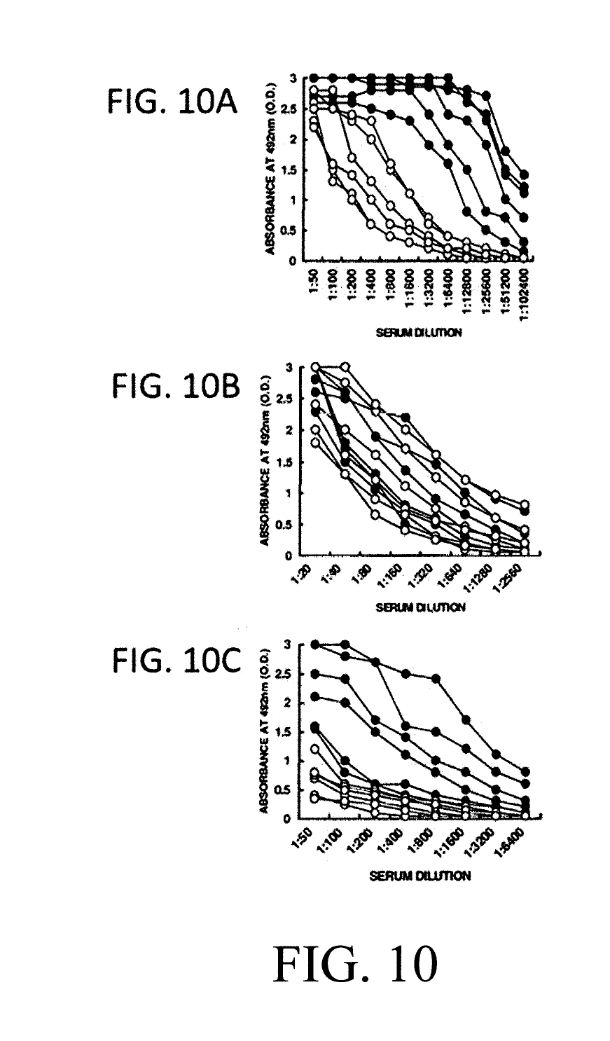

The ELISPOT results described above for influenza virus specific T cells in mice immunized with PR8 or PR8_{αgal} were validated by an independent assay that evaluates both CD8+ T cells (CTL precursors) and CD4+ T cells (Th1 helper T cells) using intracellular cytokine staining (ICS). The ICS methods utilized involved the detection of IFNγ production in activated T cells that were also stained with CD8 or CD4 specific antibodies. The spleen lymphocytes from immunized mice were co-incubated for 24 h with dendritic cells that process PR8 proteins (due to pulsing with PR8) as in the ELISPOT assays above. However, cytokine secretion was prevented by treatment with brefeldin. Subsequently, the cells were washed, permeabilized and stained for intracellular IFNγ using a labeled anti-IFNγ antibody and an anti-CD8 or an anti-CD4 antibody (Abdel-motal et al. J Virol supra 2007). As shown in FIG. 10A, only 2.6-4.4% of CD8+ T cells from PR8 immunized mice were primed by PR8 pulsed dendritic cells and thus were only marginally activated. In contrast, in 4 mice immunized with PR8_{αgal} (#1-#4), as many as 19.5-23.3% of CD8+ T cells were activated by PR8 pulsed dendritic cells. The two mice (#5 and #6) that displayed low ELISPOT values as described in the FIG. 9, also displayed low ICS levels in CD8+ T cells.

The differential response of T cells to the PR8 peptides presented by dendritic cells was also observed among the CD4+ T cells. Four of the mice immunized with PR8_{αgal} displayed 12-13.7% activation of CD4+ T cells, whereas no significant activation of such cells was observed among CD4+ T cells from PR8 immunized mice (FIG. 10B). CD4+ T cells activated to produce IFNγ represent the PR8 specific T helper Th1 cell population. The two PR8_{αgal} immunized mice (#5 and #6) with low levels of CD8+ activation, also had low levels of CD4+ activation, indicating that there was no measurably increased anti-virus cellular immune response in these mice as determined by ICS. As in the ELISPOT studies above, the ICS studies indicate that influenza virus processed to express α-gal epitopes is much more immunogenic than influenza virus lacking α-gal epitopes because of the anti-Gal binding to these epitopes and targeting of the virus by this antibody to APC via Fc/Fc receptor interaction.

Anti-Gal Mediated Increased Production of Anti-Influenza Virus Antibodies Following Immunization with Virus Expressing α-Gal Epitopes—

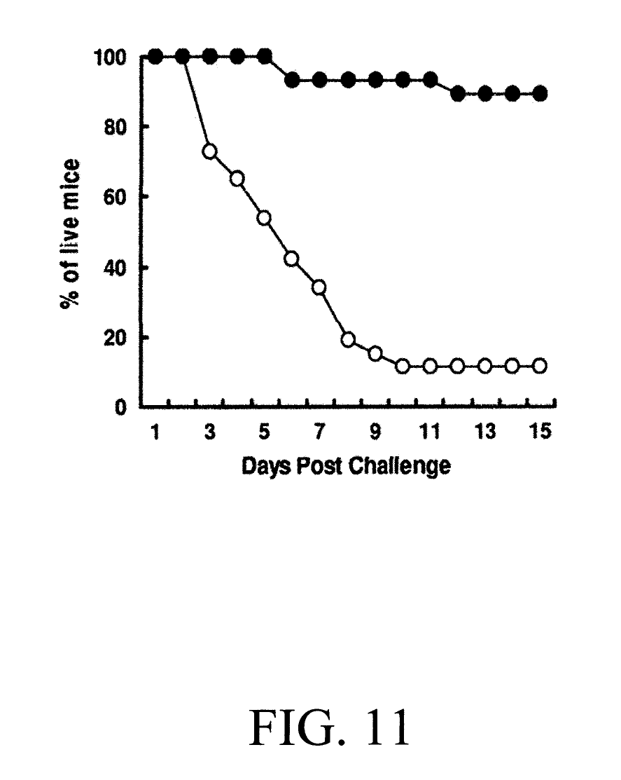

In order to evaluate anti-influenza virus antibody production in mice immunized with inactivated influenza virus, the sera from GT-KO mice immunized with PR8 or PR8_{αgal} virus were assayed for antibodies to the unprocessed PR8 virus used as solid phase antigen in ELISA. As shown in FIG. 11A the anti-PR8 IgG antibody activity in the 6 mice immunized with inactivated PR8_{αgal} virus presenting α-gal epitopes was much higher than in mice immunized with PR8 virus lacking α-gal epitopes (called PR8 virus). The four mice immunized with PR8_{αgal} that showed very high anti-PR8 antibody activity (mice #1-#4 in FIGS. 9 and 10) displayed an average of 50% maximum binding to the ELISA wells (e.g., ˜1.5 OD) at the serum dilution of 1:102,400. Even in mice #5 and #6, which displayed low levels of CD4+ and CD8+ activation, displayed 50% maximum anti-PR8 IgG activity at serum dilution of 1:12,800 and 1:6,400, respectively. In contrast, in mice immunized with inactivated PR8 virus (i.e., virus lacking α-gal epitopes), the 50% maximum binding was observed in serum dilution of only 1:400 (i.e., >200 fold lower than in PR8_{αgal} immunized mice #1-#4).

To determine whether the differences in antibody responses observed in the PR8 or PR8_{αgal} immunized GT-KO mice are dependent on the presence of the anti-Gal antibody, C57BL/6 wild type (WT) mice were also immunized with PR8 or PR8_{αgal}. The WT mice, which are the parental mice for GT-KO mice, express α-gal epitopes on their cells and thus, do not produce the anti-Gal antibody despite repeated immunizations with pig kidney membranes (PKM) (FIG. 7). As shown in FIG. 11B, no significant differences in anti-PR8 antibody responses were observed between PR8 and PR8_{αgal} immunized WT mice. Thus, in the absence of anti-Gal in WT mice, expression of α-gal epitopes on the immunizing virus has no measurable effect on the immunogenicity of the virus. Thus in WT mice (FIG. 11B) immunogenicity of PR8_{αgal} was much lower than in GT-KO mice immunized with inactivated PR8_{αgal} virus (FIG. 11A).

The differential humoral immune response (i.e. anti-virus antibody response) in GT-KO mice immunized with PR8_{αgal} versus that in GT-KO mice immunized with PR8 virus is also evident by analysis of anti-PR8 IgA antibodies in an ELISA employing PR8 virus as a solid phase antigen. The significance of the IgA immunoglobulin class is primarily in mucosal immunity that prevents viral infection of respiratory tract cells. As shown in FIG. 11C, in PR8_{αgal} immunized mice #1-#4 anti-PR8 IgA activity was 50-100 fold higher than that observed in the PR8 immunized mice #7-#12 (mice numbered in FIGS. 9 and 10). The anti-PR8 antibody studies indicate that the immunizing influenza virus carrying α-gal epitopes is much more immunogenic than immunizing influenza virus lacking α-gal epitopes. Thus, immunization with influenza_{αgal} virus induces more potent humoral as well as cellular immune responses in recipients possessing anti-Gal antibodies. It is contemplated therefore that the immunogenicity of influenza virus bound to α-gal/SA liposomes is much higher than that of unbound influenza virus because of the anti-Gal mediated increased targeting to APC of the virus bound to the α-gal/SA liposomes.

Induction of a Protective Immune Response Against Challenge with Live PR8 Influenza Virus—

The studies in this section determine whether the increased cellular and humoral immunogenicity of PR8_{αgal} virus, described above, further elevates the resistance of GT-KO mice to challenge (i.e. infection) with live PR8 virus. For this purpose, anti-Gal producing GT-KO mice were immunized twice with 1 μg of heat inactivated PR8 or PR8_{αgal} virus in the Ribi© adjuvant at two week interval. Four weeks after the second immunization, the mice were studied for resistance to challenge with 2000 plaque forming units (PFU) of live PR8 virus administered in 50 μl via the nostrils (i.e. intranasal). Each group included 26 mice. The mice were monitored for mortality every day for 30 days post challenge. Most mice (89%) immunized with inactivated PR8 virus were not resistant to the intranasal viral challenge and died within 10 days post challenge with the live PR8 virus, i.e., only 11% of the mice survived 10 days post challenge (FIG. 12). In contrast, mice immunized with inactivated PR8_{αgal} virus were much more resistant to the live virus challenge since only 11% of the mice succumbed to the live virus infection and died, whereas 89% of the mice survived the challenge (FIG. 12). These studies indicate that the heightened immune response induced by immunization of GT-KO mice with inactivated PR8_{αgal} virus is physiologically significant in that it is associated with marked decrease in mortality (i.e., increased resistance) after influenza virus challenge with a lethal dose of the virus.

Although knowledge of the mechanism(s) involved is not required in order to make and use the present invention, it is contemplated that similar to the immunological effects of anti-Gal binding to α-gal epitopes on PR8_{αgal} influenza virus, also anti-Gal binding to α-gal epitopes on α-gal/SA liposomes results in increase in immunogenicity of influenza virus that is bound to SA epitopes on α-gal/SA liposomes (as partly illustrated in FIG. 2). This is since binding of the anti-Gal antibody to the α-gal epitopes on α-gal/SA liposomes activates complement and thus mediates recruitment of macrophages and dendritic cells to these liposomes. The interaction between the Fc portion of anti-Gal bound to α-gal epitopes on PR8_{αgal} virus increases the uptake, transport, processing and presentation of the immunogenic influenza virus peptides for the activation of the corresponding influenza virus specific CD4+ and CD8+ T cells, as shown in Example 4. α-Gal epitopes on α-gal/SA liposomes have the same Galα1-3Galβ1-4GlcNAc-R structure as those on PR8_{αgal} virus. Therefore, it is contemplated that interaction between the Fc portion of anti-Gal bound to α-gal epitopes on α-gal/SA liposomes and Fc receptors on macrophages and dendritic cells results in a similar increased uptake, transport, processing and presentation of the influenza virus immunogenic peptides as that observed with PR8_{αgal} virus. This further implies that the virus peptides processed and presented following the uptake by APC of influenza virus bound to the α-gal/SA liposomes, will induce a very effective activation of influenza virus specific T cells within draining lymph nodes. These activated T cells differentiate into many cytotoxic T cells (CTL) that kill virus infected cells and thus stop the spread of the virus from on cell to the other. In addition, many influenza specific CD4+ helper T cells are activated by this process and effectively help influenza virus specific B cells to produce high titers of IgA and IgG antibodies against the virus. These antibodies neutralize the virus and prevent it from further infecting cells in the respiratory tract. It is therefore contemplated that the effective destruction of the influenza virus as a result of effective anti-Gal mediated uptake of influenza virus bound to α-gal/SA liposomes by macrophages and dendritic cells and the combined cellular and humoral immune responses against the infective influenza virus, all occur following inhalation of α-gal/SA liposomes by symptomatic influenza patients. These increased cellular and humoral immune responses stop the progression of influenza virus spread in the respiratory tract earlier than in the absence of the treatment described in this invention. Thus, this treatment shortens the period of the influenza disease and decreases the morbidity and mortality following influenza virus infection.

All publications and patents mentioned in the above specification are herein incorporated by reference. Various modifications and variations of the described method and system of the invention will be apparent to those skilled in the art without departing from the scope and spirit of the invention. Such is the use of α-gal liposomes expressing receptors for other respiratory viruses. The use of liposomes presenting epitopes that interact with natural antibodies other than α-gal epitopes, such as, but not limited to liposomes presenting rhamnose epitopes and binding natural anti-rhamnose antibodies to such liposomes, may also be contemplated for uses described in this invention for α-gal liposomes. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

### REFERENCES USED IN THIS APPLICATION

Abdel-Motal U M, Guay H M, Wigglesworth K, Welsh R M, Galili U. Increased immunogenicity of influenza virus vaccine by anti-Gal mediated targeting to antigen presenting cells. J. Virol. 81: 9131-9141. 2007.
Baum L G, Paulson J C. Sialyloligosaccharides of the respiratory epithelium in the selection of human influenza virus receptor specificity. Acta Histochem Suppl. 40:35-38, 1990.
Bovin N V. Natural antibodies to glycans. Biochemistry (Mosc). 78: 786-797, 2013.
Chen W, Gu L, Zhang W, Motari E, Cai L, Styslinger T J, Wang P G. L-rhamnose antigen: a promising alternative to α-gal for cancer immunotherapies. ACS Chem Biol. 6:185-91, 2011.
Chien J L, Li S C, Laine R A, Li Y T. Characterization of gangliosides from bovine erythrocyte membranes. J Biol Chem. 253: 4031-4035, 1978.
Cotter P F, Ayoub J, Parmentier H K. Directional selection for specific sheep cell antibody responses affects natural rabbit agglutinins of chickens. Poult Sci. 84:220-5, 2005.
Cotter P F, Van Eerden E. Natural anti-Gal and Salmonella-specific antibodies in bile and plasma of hens differing in diet efficiency. Poult Sci. 85: 435-40, 2006.
Egge H, Kordowicz M, Peter-Katalinié J, Hanfland P. Immunochemistry of I/i-active oligo- and polyglycosylceramides from rabbit erythrocyte membranes. Characterization of linear, di-, and triantennary neolactoglycosphingolipids. J Biol Chem. 260: 4927-4935, 1985.
Galili U. Evolution and pathophysiology of the human natural anti-Gal antibody. Springer Seminars in Immunopathology 15:155-171, 1993.
Galili, U. Anti-Gal: An abundant human natural antibody of multiple pathogeneses and clinical benefits. Immunology 140:1-11, 2013.
Galili U. Anti-Gal Macrophage recruitment and activation by α-gal nanoparticles accelerate regeneration and can improve biomaterials efficacy in tissue engineering. The Open Tissue Engineering and Regenerative Medicine Journal, 6: 1-11, 2013.
Galili, U. Avoiding detrimental human iImmune response against mammalian extracellular matrix implants. Tissue Engineering, Part B: Reviews, 21: 231-241, 2015.
Galili, U. Acceleration of wound healing by α-gal nanoparticles interacting with the natural anti-Gal antibody. J. Immunol. Res. Vol. 2015, Article ID 589648 April 2015
Galili, U, Rachmilewitz E A, Peleg A, Flechner I. A unique natural human IgG antibody with anti-α-galactosyl specificity. J. Exp. Med. 160: 1519-1531, 1984.
Galili, U., Macher B A. Buehler J S, Shohet B. Human natural anti-α-galactosyl IgG. II. The specific recognition of α(1→3)-linked galactose residues. J. Exp. Med. 162: 573-582, 1985.
Galili U, Clark M R, Shohet S B, Buehler J, Macher B A. Evolutionary relationship between the anti-Gal antibody and the Galα1→3Gal epitope in primates. Proc. Natl. Acad. Sci. USA. 84: 1369-1373, 1987.
Galili U, Mandrell R E, Hamadeh R M, Shohet S B, Griffiss J M. Interaction between human natural anti-α-galactosyl immunoglobulin G and bacteria of the human flora. Infect Immun. 56:1730-1737, 1988.
Galili U, Shohet S B, Kobrin E, Stults C L M, Macher B A. Man, apes, and Old World monkeys differ from other mammals in the expression of α-galactosyl epitopes on nucleated cells. J. Biol. Chem. 263; 17755-17762, 1988.
Galili U, LaTemple D C, Radic M Z. A sensitive assay for measuring α-gal epitope expression on cells by a monoclonal anti-Gal antibody. Transplantation, 65:1129-1132, 1998.
Galili U, Wigglesworth K, Abdel-Motal U M Intratumoral injection of α-gal glycolipids induces xenograft-like destruction and conversion of lesions into endogenous vaccines. J Immunol. 178: 4676-4687, 2007.
Galili U, Wigglesworth K, Abdel-Motal U M. Accelerated healing of skin burns by anti-Gal/α-gal liposomes interaction. Burns 36:239-251, 2010.
Hurwitz Z M, Ignotz R, Lalikos J F, Galili U. Accelerated porcine wound healing after treatment with α-gal nanoparticles. Plast Rcconstr Surg. 129: 242e-251e, 2012.
“Influenza (Seasonal) Fact sheet No 211”. who.int. March 2014.
Ioannidis, L. J., Verity, E. E., Crawford, S., Rockman, S. P. & Brown, L. E. Abortive replication of influenza virus in mouse dendritic cells. J Virol 86, 5922-5925, 2012.
Lai L, Kolber-Simonds D, Park K W, Cheong H T, Greenstein J L, Im G S, Samuel M, Bonk A, Rieke A, Day B N, Murphy C N, Carter D B, Hawley R J, Prather R S. Production of α-1,3-galactosyltransferase knockout pigs by nuclear transfer cloning. Science 295:1089-1092, 2002.
LaTemple, D. C. and U. Galili. Adult and neonatal anti-Gal response in knock-out mice for α-galactosyltransferase. Xenotransplantation, 5:191-196, 1998.
Matrosovich M, Klenk H D. Natural and synthetic sialic acid-containing inhibitors of infuenza virus receptor binding. Rev Med Virol. 13: 85-97, 2003
McKenzie I F, Patton K, Smit J A, Mouhtouris E, Xing P, Myburgh J A, Sandrin M S. Definition and characterization of chicken Gal alpha(1,3)Gal antibodies. Transplantation. 67:864-70, 1999.
Minozzi G, Parmentier H K, Mignon-Grasteau S, Nieuwland M G, Bed′hom B, Gourichon D, Minvielle F, Pinard-van der Laan M H. Correlated effects of selection for immunity in White Leghorn chicken lines on natural antibodies and specific antibody responses to KLH and M. butyricum. BMC Genet. 14; 9:5, 2008.
Mochalova L, Gambaryan A, Romanova J, Tuzikov A, Chinarev A, Katinger D, Katinger H, Egorov A, Bovin N. Receptor-binding properties of modern human influenza viruses primarily isolated in Vero and MDCK cells and chicken embryonated eggs. Virology 313: 473-480, 2003.
Okusanya O O, Bhavnani S M, Hammel J P, Forrest A, Bulik C C, Ambrose P G, Gupta R. Evaluation of the pharmacokinetics and pharmacodynamics of liposomal amikacin for inhalation in cystic fibrosis patients with chronic pseudomonal infections using data from two phase 2 clinical studies. Antimicrob Agents Chemother. 58: 5005-15, 2014.
Olofsson S, Bergström T. Glycoconjugate glycans as viral receptors. Ann Med. 37: 154-172, 2005.
Oshansky C M, Pickens J A, Bradley K C, Jones L P, Saavedra-Ebner G M, Barber J P, Crabtree J M, Steinhauer D A, Tompkins S M, Tripp R A. Avian influenza viruses infect primary human bronchial epithelial cells unconstrained by sialic acid α2,3 residues. PLoS One. 6:e21183, 2011
Peschke T, Bender A, Nain M, Gemsa D. Role of macrophage cytokines in influenza A virus infections. Immunobiology. 189: 340-355, 1993.
Phelps C J, Koike C, Vaught T D, Boone J, Wells K D, Chen S H, Ball S, Specht S M, Polejaeva I A, Monahan J A, Jobst P M, Sharma S B, Lamborn A E, Garst A S, Moore M, Demetris A J, Rudert W A, Bottino R, Bertera S, Trucco M, Starzl T E, Dai Y, Ayares D L. Production of α1,3-galactosyltransferase-deficient pigs. Science. 17: 299: 411-414, 2002.
Reading P C, Miller J L, Anders E M. Involvement of the mannose receptor in infection of macrophages by influenza virus. J Virol 74: 5190-5197, 2000.
Rogers G N, Paulson J C. Receptor determinants of human and animal influenza virus isolates: differences in receptor specificity of the H3 hemagglutinin based on species of origin. Virology. 127: 361-373, 1983.
Sandrin M S, Vaughan H A, Xing P X, McKenzie I F. Natural human anti-Gal α(1,3)Gal antibodies react with human mucin peptides. Glycoconj J. 14: 97-105, 1997.
Schirmer J M, Fass D N, Byrne G W, Tazelaar H D, Logan J S, McGregor C G. Effective antiplatelet therapy does not prolong transgenic pig to baboon cardiac xenograft survival. Xenotransplantation. 11: 436-443, 2004.
Simon P M, Neethling F A, Taniguchi S, Goode P L, Zopf D, Hancock W W, Cooper D K. Intravenous infusion of Gal α1-3Gal oligosaccharides in baboons delays hyperacute rejection of porcine heart xenografts. Transplantation. 1998 Feb. 15; 65(3):346-53.
Skehel J J, Wiley D C. Receptor binding and membrane fusion in virus entry: the influenza hemagglutinin. Annu Rev Biochem 69, 531-569, 2000.
Siekmeier R, Scheuch G. Inhaled insulin—does it become reality? J Physiol Pharmacol. 59: 81-113, 2008.
Stone K R, Abdel-Motal U M, Walgenbach A W, Turek T J, Galili U. Replacement of human anterior cruciate ligaments with pig ligaments: a model for anti-non-gal antibody response in long-term xenotransplantation. Transplantation. 83: 211-219, 2007.
Suzuki Y, Nagao Y, Kato H, Matsumoto M, Nerome K, Nakajima K, Nobusawa E. Human influenza A virus hemagglutinin distinguishes sialyloligosaccharides in membrane-associated gangliosides as its receptor which mediates the adsorption and fusion processes of virus infection. Specificity for oligosaccharides and sialic acids and the sequence to which sialic acid is attached. J Biol Chem. 1986 261: 17057-17061
Tearle R G, Tange M J, Zannettino Z L, Katerelos M, Shinkel T A, Van Denderen B J, Lonie A J, Lyons I, Nottle M B, Cox T, Becker C, Peura A M, Wigley P L, Crawford R J, Robins A J, Pearse M J, d'Apice A J. The α-1,3-galactosyltransferase knockout mouse. Implications for xenotransplantation. Transplantation. 61: 13-19, 1996.
Thall A D, Malý P, Lowe J B. Oocyte Gal α1,3Gal epitopes implicated in sperm adhesion to the zona pellucida glycoprotein ZP3 are not required for fertilization in the mouse. J Biol Chem. 270: 21437-21440, 1995.
Thompson C I, Barclay W S, Zambon M C, Pickles R J. Infection of human airway epithelium by human and avian strains of influenza A virus. J Virol 80: 8060-8068, 2006.
Uemura K, Yuzawa M, Taketomi T. Characterization of major glycolipids in bovine erythrocyte membrane. J Biochem. 83:463-471, 1978.
Unverzagt C, Kelm S, Paulson J C. Chemical and enzymatic synthesis of multivalent sialoglycopeptides. Carbohydr Res. 251: 285-301, 1994.
Watanabe K, Hakomori S I, Childs R A, Feizi T. Characterization of a blood group I-active ganglioside. Structural requirements for I and i specificities. J Biol Chem 254: 3221-3228, 1979.
Webster R G. Immunity to influenza in the elderly., Vaccine, 18:1686-1689, 2000.
Wigglesworth K M, Racki W J, Mishra R, Szomolanyi-Tsuda E, Greiner D L, Galili U. Rapid recruitment and activation of macrophages by anti-Gal/α-Gal liposome interaction accelerates wound healing. J Immunol. 186: 4422-4432, 2011.
Wiley D C, Skehel J J. The structure and function of the hemagglutinin membrane glycoprotein of influenza virus. Annu Rev Biochem 56: 365-394, (1987).
Xu Y, Lorf T, Sablinski T, Gianello P, Bailin M, Monroy R, Kozlowski T, Awwad M, Cooper D K, Sachs D H. Removal of anti-porcine natural antibodies from human and nonhuman primate plasma in vitro and in vivo by a Galα1-3Galβ1-4βGlc-X immunoaffinity column. Transplantation. 65: 172-179, 1998.

### Patents Pertinent to this Application and Preceding it (Uri Galili)

1. Compositions and methods for vaccines comprising α-galactosyl epitopes U.S. Pat. No. 5,879,675, Issued: Mar. 9, 1999.
2. Compositions and methods for vaccines comprising α-galactosyl epitopes, U.S. Pat. No. 6,361,775, Issued: Mar. 26, 2002.
3. Compositions and methods for wound healing, U.S. Pat. No. 8,084,057, Issued: Dec. 27, 2011.
4. Compositions and methods for wound healing, U.S. Pat. No. 8,440,198, Issued: May 14, 2013.
5. Compositions and methods for wound healing, U.S. Pat. No. 8,865,178, Issued: Oct. 21, 2014.

We claim:

1. A method for treating respiratory diseases caused by an infectious microbial agent in an animal having endogenous natural antibody, comprising administering by inhalation of liposomes that present both binding receptors to said infectious agent and ligands to said natural antibody wherein:
a) said inhaled liposomes land in mucus and surfactant films coating the respiratory tract epithelium and bind said infectious agent by receptors to said infectious agent on said liposomes,
b) inhalation of said liposomes is under conditions such that binding of infectious agent to corresponding receptor on said liposomes inhibits further infection of respiratory tract epithelium by said infectious agent,
c) inhalation of said liposomes is under conditions such that binding of said natural antibody to said ligands on said inhaled liposomes induces recruitment of granulocytes, monocytes, macrophages and dendritic cells into the respiratory tract of said animal,
d) natural antibody bound to said inhaled liposomes induces internalization of said infectious agent bound to said liposomes into granulocytes, monocytes, macrophages and dendritic cells, and
e) said infectious agent infectious agent internalized into monocytes, macrophages and dendritic cells is processed by these cells to become immunogenic peptides that are transported by these cells to lymph nodes and spleen under conditions that immunogenic peptides induce an effective, protective immune response against said infectious agent.

2. The method of claim 1, wherein:
a) said ligand binding said natural antibody is selected from the group consisting of terminal non-reducing galactose, glucose, rhamnose, mannose, fucose, N-acetyl-glucosamine, N-acetyl-galactosamine, sialic acid that is N-acetyl-neuraminic acid or N-glycolyl-neuraminic acid,
b) said ligand binding natural antibody and said infectious agent binding receptor on said inhaled liposomes are linked directly or by a linker to a molecule in the liposomes wall which is selected from the group consisting of glycolipids, glycoproteins, proteoglycans, polymers, lipids, or proteins.

3. The method of claim 1, wherein said natural antibody binding to the inhaled liposomes is the natural anti-Gal antibody and the ligand on the inhaled liposomes that binds the natural anti-Gal antibody is the α-gal epitope, or any epitope capable of binding said natural anti-Gal antibody.

4. The method in claim 1 in which the ligand on said liposomes for an antibody is immunocomplexed with the corresponding antibody prior to administration by inhalation of said liposomes to treated animal.

5. The method of claim 1, wherein said animal is selected from the group consisting of birds, mammals and humans.

6. The method of claim 1 wherein receptors to said infectious agent and said ligand to endogenous natural antibody are both linked to a biodegradable particulate material, or to a molecule from the group of proteins, lipids, proteoglycans, or polymers and used for treatment by inhalation similar to the use of said liposomes.

7. The method of claims 1, 2, 3 and 4, for the treatment of animals infected in the respiratory tract with influenza virus, wherein:
a) said infectious agent is influenza virus,
b) said infectious agent binding receptor on said inhaled liposomes is selected from the group consisting N-acetyl-neuraminic acid, or N-glycolyl-neuraminic acid, both referred to as sialic acid,
c) said endogenous natural antibody is the anti-Gal antibody, and
d) said ligand to the natural anti-Gal antibody presented on said inhaled liposomes is a glycolipid having a non-reducing end that comprises an α-gal epitope comprising galactosyl α1-3galactosyl, or any other epitope that is capable of binding the natural anti-Gal antibody.

8. The method in claim 7 for treating a subject infected with influenza virus having endogenous anti-Gal antibody by inhalation of a biodegradable composition of liposomes which present both α-gal epitopes and sialic acid epitopes wherein:
a) influenza virus infecting the respiratory tract binds to said sialic acid epitopes on said inhaled liposomes and is prevented from infecting respiratory epithelium cells,
b) inhalation of said liposomes is under conditions such that the natural anti-Gal antibody binds to said α-gal epitopes on said inhaled liposomes in the respiratory tract,
c) interaction between the natural anti-Gal antibody and α-gal epitopes on said inhaled liposomes is under conditions such that induce recruitment of granulocytes, monocytes, macrophages and dendritic cells to said liposomes,
d) the said recruited granulocytes, monocytes, macrophages and dendritic cells internalize said liposomes and the influenza virus bound to said liposomes,
e) said influenza virus internalized into monocytes, macrophages and dendritic cells is processed by these cells to become immunogenic peptides that are transported to lymph nodes and spleen and that induce an effective, protective immune response against influenza virus.

9. A method in claims 7 and 8 wherein the treated subject is a human.

10. A method in claims 7 and 8 wherein the treated subject is selected from groups of apes, Old World monkeys, or birds.
